(19) United States
(12) Patent Application Publication
(10) Pub. No.: US 2024/0361736 A1
(43) Pub. Date: Oct. 31, 2024

(54) SMART ROOM FOR A HEALTHCARE FACILITY

(71) Applicant: Johnson Controls Tyco IP Holdings LLP, Milwaukee, WI (US)

(72) Inventors: Julie J. Brown, Yardley, PA (US); Brendon F. Buckley, Carmel, IN (US)

(21) Appl. No.: 18/560,362

(22) PCT Filed: May 11, 2022
(86) PCT No.: PCT/US2022/028840

§ 371 Date: Nov. 10, 2023

Related U.S. Application Data
(60) Provisional application No. 63/187,836, filed on May 12, 2021.

(51) Int. Cl. G05B 15/02; G16H 10/60; G16H 40/20

(52) U.S. Cl. CPC G05B 15/02; G16H 10/60; G16H 40/20; G05B 2219/2642

(57) ABSTRACT

A building management system (BMS) of a building for controlling a healthcare facility. The BMS including one or more processing circuits comprising one or more memory devices configured to store instructions thereon that, when executed by one or more processors, cause the one or more processors to: receive inputs from building devices; determine a state-of-mind (SoM) score of a patient based on the inputs using a learning model; and control one or more building devices based on the SoM score while maintaining a temperature, a humidity, and a pressure within a compliance standard within the building.

### CROSS-REFERENCE TO RELATED APPLICATIONS

This application claims priority to U.S. Provisional Patent Application No. 63/187,836, filed May 12, 2021, the entire contents of which are incorporated by reference herein.

### BACKGROUND

The present disclosure generally relates to smart building systems. A building can include a building management system (BMS) which is a system of devices configured to control, monitor, and manage equipment in or around a building or building area. A BMS can include, for example, a HVAC system, a security system, a lighting system, a fire safety system, any other system that is capable of managing building functions or devices, or any combination thereof. Additionally, buildings can include various other systems or devices, sometimes interconnected with the BMS, to provide or enhance building automation. In some cases, such as in a healthcare facility, a smart building and/or smart building systems can improve an occupant's overall experience, by improving occupant comfort, automating various tasks, learning occupant preferences, etc.

### SUMMARY

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

One implementation of the present disclosure is a building management system (BMS) of a building for controlling a healthcare facility. The BMS including one or more processing circuits comprising one or more memory devices configured to store instructions thereon that, when executed by one or more processors, cause the one or more processors to: receive inputs from building devices; determine a state-of-mind (SOM) score of a patient based on the inputs using a learning model; and control one or more building devices based on the SOM score while maintaining a temperature, a humidity, and a pressure within a compliance standard within the building.

Another implementation of the present disclosure is a system for providing smart functionality within a room of a healthcare facility. The system includes a sensor array configured to detect occupancy within the room, measure physiological parameters of occupants of the room, and measure climate parameters of the room, and one or more processing circuits comprising one or more memory devices coupled to one or more processors, the one or more memory devices configured to store instructions thereon that, when executed by the one or more processors, cause the one or more processors to: continuously monitor a comfort level of a patient associated with the room by determining, based on first data received from the sensor array, the comfort level of the patient, comparing the determined comfort level of the patient with a threshold, and controlling one or more climate control devices associated with the room based on a determination that the comfort level of the patient exceeds the threshold; determine a healthcare schedule and a visitor schedule for the patient; and adjust at least one of a scheduled meal time or lighting within the room based on the healthcare schedule and the visitor schedule.

Another implementation of the present disclosure is a system for providing smart functionality within a room of a healthcare facility. The system includes a sensor array configured to detect occupancy within the room, measure physiological parameters of occupants of the room, and measure climate parameters of the room, and one or more processing circuits comprising one or more memory devices coupled to one or more processors, the one or more memory devices configured to store instructions thereon that, when executed by the one or more processors, cause the one or more processors to: continuously monitor a comfort level of a patient associated with the room by determining, based on first data received from the sensor array, the comfort level of the patient, comparing the determined comfort level of the patient with a threshold, and controlling one or more climate control devices associated with the room based on a determination that the comfort level of the patient exceeds the threshold; determine a healthcare schedule and a visitor schedule for the patient; and adjust at least one of a scheduled meal time or lighting within the room based on the healthcare schedule and the visitor schedule.

Another implementation of the present disclosure is a building management system (BMS) includes a plurality of security devices positioned at one or more entrances of a building, wherein at least a portion of the security devices are configured to capture image data; and one or more processing circuits comprising one or more memory devices coupled to one or more processors, the one or more memory devices configured to store instructions thereon that, when executed by the one or more processors, cause the one or more processors to: receive first image data from the plurality of security devices, analyze the first image data to identify a person entering the building, determine an appointment time for the person, initiate a check-in procedure for the person based on the identification of the person and a determination that the person has an upcoming appointment, and in response to completing the check-in procedure, transmit a notification to a user device associated with the person, the notification providing a plurality of appointment information.

Another implementation of the present disclosure is a method of providing navigation and parking to a user. The method includes receiving, from a user device associated with the user, a first request for navigation to a target location, the first request received in response to determination that the user has an upcoming appointment at the target location; determining a current location of the user based on a location of the user device; generating a first route from the current location to the target location; receiving, from a user device associated with the user, a second request to reserve parking at the target location; identifying an available parking spot based on a time period and a location of the user's upcoming appointment; generating a token to provide access to the available parking spot; and transmitting the token to the user device, wherein the user device is configured to display the token via a user interface.

### BRIEF DESCRIPTION OF THE DRAWINGS

Objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1 is a drawing of a building equipped with a HVAC system, according to some embodiments.

FIG. 2 is a block diagram of a waterside system that may be used in conjunction with the building of FIG. 1, according to some embodiments.

FIG. 3 is a block diagram of an airside system that may be used in conjunction with the building of FIG. 1, according to some embodiments.

FIG. 4 is a block diagram of a building management system (BMS) that may be used to monitor and/or control the building of FIG. 1, according to some embodiments.

FIG. 5 is a diagram of a site that includes the building of FIG. 1, according to some embodiments.

FIG. 6 is a block diagram of a control system for a smart building, such as the building of FIG. 1, according to some embodiments.

FIG. 7 is a block diagram of a central computing system for a smart building, such as the building of FIG. 1, according to some embodiments.

FIG. 8 is a diagram of a smart room that can be included in the building of FIG. 1, according to some embodiments.

FIG. 9 is a block diagram of a user device that includes a smart room control application, according to some embodiments.

FIGS. 10A and 10B are flow diagrams of processes for providing navigation and parking data to a user device, according to some embodiments.

FIGS. 11A-11D are example interfaces for presenting navigation and parking data, according to some embodiments.

FIG. 12 is a flow diagram of a process for automatic check-in based on facial recognition, according to some embodiments.

FIGS. 13A and 13B are example interfaces for an automatic check-in process, according to some embodiments.

FIGS. 14A-14C are example interfaces for various smart room functions, according to some embodiments.

FIGS. 15 and 16 are an example interfaces for controlling visitor access to a smart room, according to some embodiments.

### DETAILED DESCRIPTION

Referring generally to the FIGURES, a system and methods for implementing and controlling smart building systems are shown. More specifically, a control system for a so-called “smart” building or for a building with smart (i.e., sentient) rooms can include a central computing system, remote systems and devices, and a building management system (BMS) that, together, function to control various aspects of smart rooms or spaces within the building. User devices (e.g., mobile devices) can be used to interact with the control system, such as to view and modify information (e.g., schedules), control smart room functions (e.g., lighting, temperature, etc.), control building or room access, and to perform various other functions as discussed in detail below.

In some embodiments, a user device can implement an application or a program that allows a user of the device to interact with the smart building's various systems and devices. In a hospital setting, for example, the application may allow the user to check-in for an appointment, view a schedule, request or change an appointment time, view hospital staff information, etc. Once in a smart room, the user (e.g., a patient) may be able to control various room parameters, such as temperature, humidity, lighting, blinds, etc. Additionally, the user may be able to determine a route to the hospital and/or reserve parking via the application, and the application may also provide navigation throughout the hospital complex. Various other features of the above-mentioned application and the smart building systems are discussed in greater detail below.

Building with Building Systems

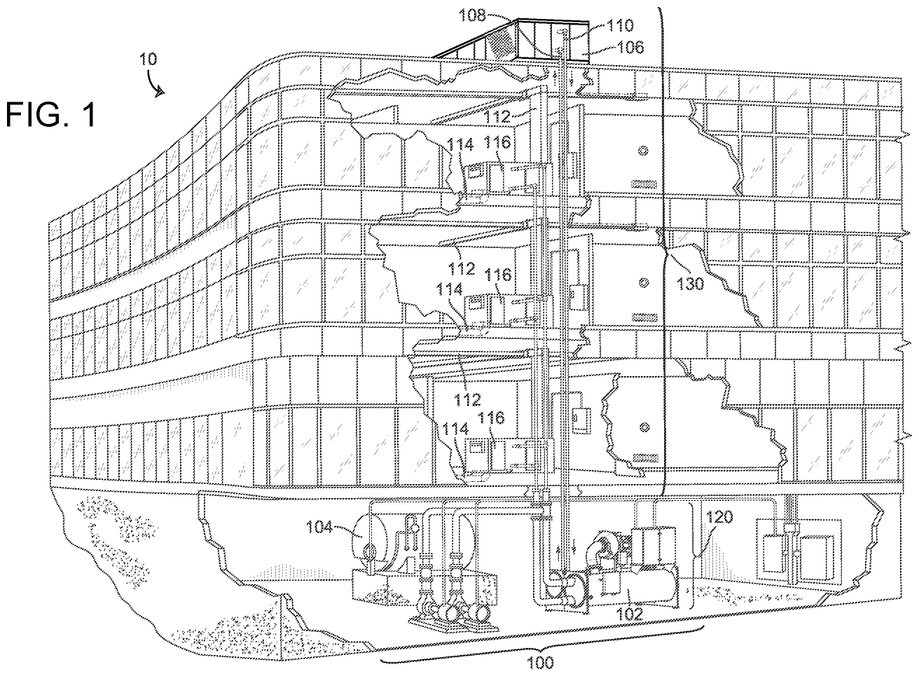

Referring now to FIGS. 1-4, an exemplary building management system (BMS) and HVAC system in which the systems and methods of the present disclosure can be implemented are shown, according to some embodiments. Referring particularly to FIG. 1, a perspective view of a building 10 (e.g., a hospital or healthcare facility) is shown. Building 10 is served by a BMS. A BMS is, in general, a system of devices configured to control, monitor, and manage equipment in or around a building or building area. A BMS can include, for example, a HVAC system, a security system, a lighting system, a fire safety system, any other system that is capable of managing building functions or devices, or any combination thereof.

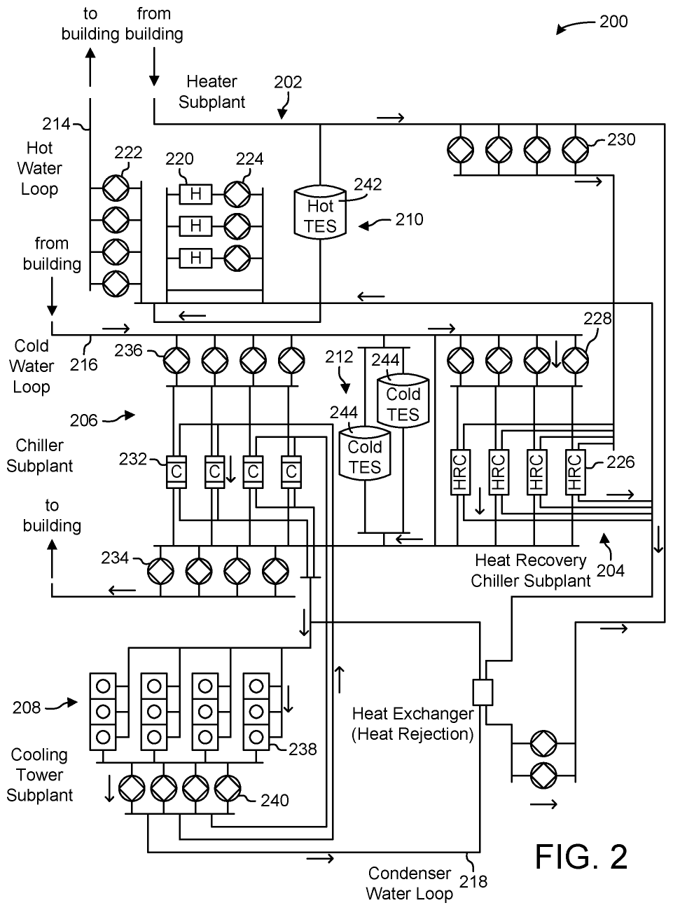

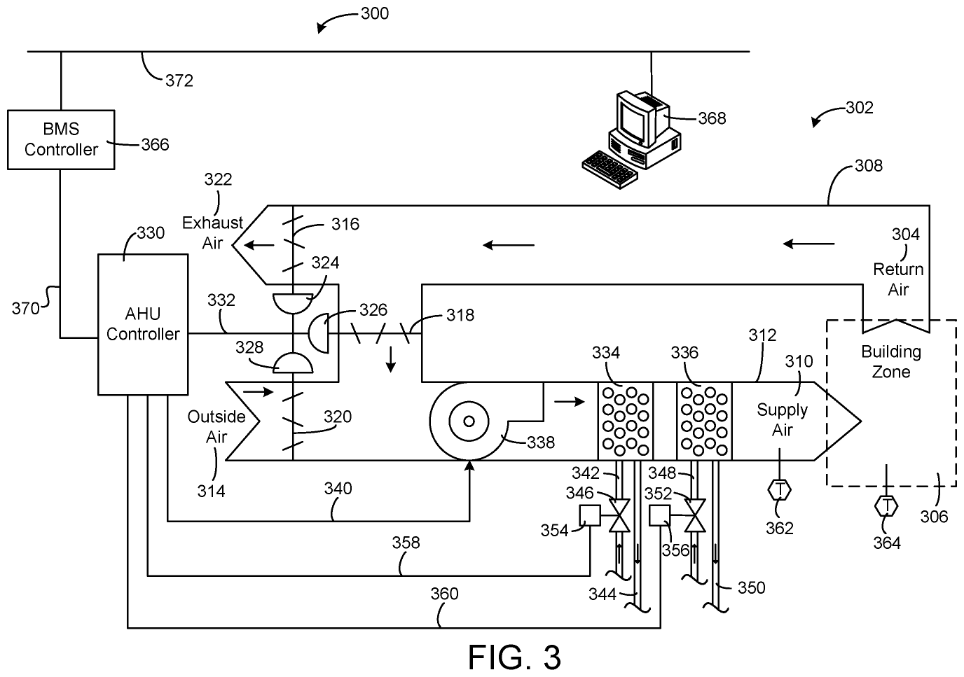

The BMS that serves building 10 includes an HVAC system 100. HVAC system 100 can include a plurality of HVAC devices (e.g., heaters, chillers, air handling units, pumps, fans, thermal energy storage, etc.) configured to provide heating, cooling, ventilation, or other services for building 10. For example, HVAC system 100 is shown to include a waterside system 120 and an airside system 130. Waterside system 120 can provide a heated or chilled fluid to an air handling unit of airside system 130. Airside system 130 can use the heated or chilled fluid to heat or cool an airflow provided to building 10. An exemplary waterside system and airside system which can be used in HVAC system 100 are described in greater detail with reference to FIGS. 2-3.

HVAC system 100 is shown to include a chiller 102, a boiler 104, and a rooftop air handling unit (AHU) 106. Waterside system 120 can use boiler 104 and chiller 102 to heat or cool a working fluid (e.g., water, glycol, etc.) and can circulate the working fluid to AHU 106. In embodiments, the HVAC devices of waterside system 120 can be located in or around building 10 (as shown in FIG. 1) or at an offsite location such as a central plant (e.g., a chiller plant, a steam plant, a heat plant, etc.). The working fluid can be heated in boiler 104 or cooled in chiller 102, depending on whether heating or cooling is required in building 10. Boiler 104 can add heat to the circulated fluid, for example, by burning a combustible material (e.g., natural gas) or using an electric heating element. Chiller 102 can place the circulated fluid in a heat exchange relationship with another fluid (e.g., a refrigerant) in a heat exchanger (e.g., an evaporator) to absorb heat from the circulated fluid. The working fluid from chiller 102 and/or boiler 104 can be transported to AHU 106 via piping 108.

AHU 106 can place the working fluid in a heat exchange relationship with an airflow passing through AHU 106 (e.g., via one or more stages of cooling coils and/or heating coils). The airflow can be, for example, outside air, return air from within building 10, or a combination of both. AHU 106 can transfer heat between the airflow and the working fluid to provide heating or cooling for the airflow. For example, AHU 106 can include one or more fans or blowers configured to pass the airflow over or through a heat exchanger containing the working fluid. The working fluid can then return to chiller 102 or boiler 104 via piping 110.

Airside system 130 can deliver the airflow supplied by AHU 106 (i.e., the supply airflow) to building 10 via air supply ducts 112 and can provide return air from building 10 to AHU 106 via air return ducts 114. In some embodiments, airside system 130 includes multiple variable air volume (VAV) units 116. For example, airside system 130 is shown to include a separate VAV unit 116 on each floor or zone of building 10. VAV units 116 can include dampers or other flow control elements that can be operated to control an amount of the supply airflow provided to individual zones of building 10. In other embodiments, airside system 130 delivers the supply airflow into one or more zones of building 10 (e.g., via supply ducts 112) without using intermediate VAV units 116 or other flow control elements. AHU 106 can include sensors (e.g., temperature sensors, pressure sensors, etc.) configured to measure attributes of the supply airflow. AHU 106 can receive input from sensors located within AHU 106 and/or within the building zone and can adjust the flow rate, temperature, or other attributes of the supply airflow through AHU 106 to achieve setpoint conditions for the building zone.

In FIG. 2, waterside system 200 is shown as a central plant having a plurality of subplants 202-212. Subplants 202-212 are shown to include a heater subplant 202, a heat recovery chiller subplant 204, a chiller subplant 206, a cooling tower subplant 208, a hot thermal energy storage (TES) subplant 210, and a cold thermal energy storage (TES) subplant 212. Subplants 202-212 consume resources (e.g., water, natural gas, electricity, etc.) from utilities to serve the thermal energy loads (e.g., hot water, cold water, heating, cooling, etc.) of a building or campus. For example, heater subplant 202 may be configured to heat water in a hot water loop 214 that circulates the hot water between heater subplant 202 and building 10. Chiller subplant 206 may be configured to chill water in a cold water loop 216 that circulates the cold water between chiller subplant 206 building 10. Heat recovery chiller subplant 204 may be configured to transfer heat from cold water loop 216 to hot water loop 214 to provide additional heating for the hot water and additional cooling for the cold water. Condenser water loop 218 may absorb heat from the cold water in chiller subplant 206 and reject the absorbed heat in cooling tower subplant 208 or transfer the absorbed heat to hot water loop 214. Hot TES subplant 210 and cold TES subplant 212 may store hot and cold thermal energy, respectively, for subsequent use.

Hot water loop 214 and cold water loop 216 may deliver the heated and/or chilled water to air handlers located on the rooftop of building 10 (e.g., AHU 106) or to individual floors or zones of building 10 (e.g., VAV units 116). The air handlers push air past heat exchangers (e.g., heating coils or cooling coils) through which the water flows to provide heating or cooling for the air. The heated or cooled air may be delivered to individual zones of building 10 to serve the thermal energy loads of building 10. The water then returns to subplants 202-212 to receive further heating or cooling.

Although subplants 202-212 are shown and described as heating and cooling water for circulation to a building, it is understood that any other type of working fluid (e.g., glycol, CO2, etc.) may be used in place of or in addition to water to serve the thermal energy loads. In other embodiments, subplants 202-212 may provide heating and/or cooling directly to the building or campus without requiring an intermediate heat transfer fluid. These and other variations to waterside system 200 are within the teachings of the present invention.

Each of subplants 202-212 may include a variety of equipment configured to facilitate the functions of the subplant. For example, heater subplant 202 is shown to include a plurality of heating elements 220 (e.g., boilers, electric heaters, etc.) configured to add heat to the hot water in hot water loop 214. Heater subplant 202 is also shown to include several pumps 222 and 224 configured to circulate the hot water in hot water loop 214 and to control the flow rate of the hot water through individual heating elements 220. Chiller subplant 206 is shown to include a plurality of chillers 232 configured to remove heat from the cold water in cold water loop 216. Chiller subplant 206 is also shown to include several pumps 234 and 236 configured to circulate the cold water in cold water loop 216 and to control the flow rate of the cold water through individual chillers 232.

Heat recovery chiller subplant 204 is shown to include a plurality of heat recovery heat exchangers 226 (e.g., refrigeration circuits) configured to transfer heat from cold water loop 216 to hot water loop 214. Heat recovery chiller subplant 204 is also shown to include several pumps 228 and 230 configured to circulate the hot water and/or cold water through heat recovery heat exchangers 226 and to control the flow rate of the water through individual heat recovery heat exchangers 226. Cooling tower subplant 208 is shown to include a plurality of cooling towers 238 configured to remove heat from the condenser water in condenser water loop 218. Cooling tower subplant 208 is also shown to include several pumps 240 confautfigured to circulate the condenser water in condenser water loop 218 and to control the flow rate of the condenser water through individual cooling towers 238.

Hot TES subplant 210 is shown to include a hot TES tank 242 configured to store the hot water for later use. Hot TES subplant 210 may also include one or more pumps or valves configured to control the flow rate of the hot water into or out of hot TES tank 242. Cold TES subplant 212 is shown to include cold TES tanks 244 configured to store the cold water for later use. Cold TES subplant 212 may also include one or more pumps or valves configured to control the flow rate of the cold water into or out of cold TES tanks 244.

In some embodiments, one or more of the pumps in waterside system 200 (e.g., pumps 222, 224, 228, 230, 234, 236, and/or 240) or pipelines in waterside system 200 include an isolation valve associated therewith. Isolation valves may be integrated with the pumps or positioned upstream or downstream of the pumps to control the fluid flows in waterside system 200. In embodiments, waterside system 200 may include more, fewer, or different types of devices and/or subplants based on the particular configuration of waterside system 200 and the types of loads served by waterside system 200.

Referring now to FIG. 3, a block diagram of an airside system 300 is shown, according to some embodiments. In embodiments, airside system 300 may supplement or replace airside system 130 in HVAC system 100 or may be implemented separate from HVAC system 100. When implemented in HVAC system 100, airside system 300 may include a subset of the HVAC devices in HVAC system 100 (e.g., AHU 106, VAV units 116, ducts 112-114, fans, dampers, etc.) and may be located in or around building 10. Airside system 300 may operate to heat or cool an airflow provided to building 10 using a heated or chilled fluid provided by waterside system 200.

In FIG. 3, airside system 300 is shown to include an economizer-type air handling unit (AHU) 302. Economizer-type AHUs vary the amount of outside air and return air used by the air handling unit for heating or cooling. For example, AHU 302 may receive return air 304 from building zone 306 via return air duct 308 and may deliver supply air 310 to building zone 306 via supply air duct 312. In some embodiments, AHU 302 is a rooftop unit located on the roof of building 10 (e.g., AHU 106 as shown in FIG. 1) or otherwise positioned to receive both return air 304 and outside air 314. AHU 302 may be configured to operate exhaust air damper 316, mixing damper 318, and outside air damper 320 to control an amount of outside air 314 and return air 304 that combine to form supply air 310. Any return air 304 that does not pass through mixing damper 318 may be exhausted from AHU 302 through exhaust damper 316 as exhaust air 322.

Each of dampers 316-320 may be operated by an actuator. For example, exhaust air damper 316 may be operated by actuator 324, mixing damper 318 may be operated by actuator 326, and outside air damper 320 may be operated by actuator 328. Actuators 324-328 may communicate with an AHU controller 330 via a communications link 332. Actuators 324-328 may receive control signals from AHU controller 330 and may provide feedback signals to AHU controller 330. Feedback signals may include, for example, an indication of a current actuator or damper position, an amount of torque or force exerted by the actuator, diagnostic information (e.g., results of diagnostic tests performed by actuators 324-328), status information, commissioning information, configuration settings, calibration data, and/or other types of information or data that may be collected, stored, or used by actuators 324-328. AHU controller 330 may be an economizer controller configured to use one or more control algorithms (e.g., state-based algorithms, extremum seeking control (ESC) algorithms, proportional-integral (PI) control algorithms, proportional-integral-derivative (PID) control algorithms, model predictive control (MPC) algorithms, feedback control algorithms, etc.) to control actuators 324-328.

Still referring to FIG. 3, AHU 302 is shown to include a cooling coil 334, a heating coil 336, and a fan 338 positioned within supply air duct 312. Fan 338 may be configured to force supply air 310 through cooling coil 334 and/or heating coil 336 and provide supply air 310 to building zone 306. AHU controller 330 may communicate with fan 338 via communications link 340 to control a flow rate of supply air 310. In some embodiments, AHU controller 330 controls an amount of heating or cooling applied to supply air 310 by modulating a speed of fan 338.

Cooling coil 334 may receive a chilled fluid from waterside system 200 (e.g., from cold water loop 216) via piping 342 and may return the chilled fluid to waterside system 200 via piping 344. Valve 346 may be positioned along piping 342 or piping 344 to control a flow rate of the chilled fluid through cooling coil 334. In some embodiments, cooling coil 334 includes multiple stages of cooling coils that can be independently activated and deactivated (e.g., by AHU controller 330, by BMS controller 366, etc.) to modulate an amount of cooling applied to supply air 310.

Heating coil 336 may receive a heated fluid from waterside system 200 (e.g., from hot water loop 214) via piping 348 and may return the heated fluid to waterside system 200 via piping 350. Valve 352 may be positioned along piping 348 or piping 350 to control a flow rate of the heated fluid through heating coil 336. In some embodiments, heating coil 336 includes multiple stages of heating coils that can be independently activated and deactivated (e.g., by AHU controller 330, by BMS controller 366, etc.) to modulate an amount of heating applied to supply air 310.

Each of valves 346 and 352 may be controlled by an actuator. For example, valve 346 may be controlled by actuator 354 and valve 352 may be controlled by actuator 356. Actuators 354-356 may communicate with AHU controller 330 via communications links 358-360. Actuators 354-356 may receive control signals from AHU controller 330 and may provide feedback signals to controller 330. In some embodiments, AHU controller 330 receives a measurement of the supply air temperature from a temperature sensor 362 positioned in supply air duct 312 (e.g., downstream of cooling coil 334 and/or heating coil 336). AHU controller 330 may also receive a measurement of the temperature of building zone 306 from a temperature sensor 364 located in building zone 306.

In some embodiments, AHU controller 330 operates valves 346 and 352 via actuators 354-356 to modulate an amount of heating or cooling provided to supply air 310 (e.g., to achieve a setpoint temperature for supply air 310 or to maintain the temperature of supply air 310 within a setpoint temperature range). The positions of valves 346 and 352 affect the amount of heating or cooling provided to supply air 310 by cooling coil 334 or heating coil 336 and may correlate with the amount of energy consumed to achieve a desired supply air temperature. AHU controller 330 may control the temperature of supply air 310 and/or building zone 306 by activating or deactivating coils 334-336, adjusting a speed of fan 338, or a combination of both.

Still referring to FIG. 3, airside system 300 is shown to include a building automation system (BMS) controller 366 and a client device 368. BMS controller 366 may include one or more computer systems (e.g., servers, supervisory controllers, subsystem controllers, etc.) that serve as system level controllers, application or data servers, head nodes, or master controllers for airside system 300, waterside system 200, HVAC system 100, and/or other controllable systems that serve building 10. BMS controller 366 may communicate with multiple downstream building systems or subsystems (e.g., HVAC system 100, a security system, a lighting system, waterside system 200, etc.) via a communications link 370 according to like or disparate protocols (e.g., LON, BACnet, etc.). In embodiments, AHU controller 330 and BMS controller 366 may be separate (as shown in FIG. 3) or integrated. In an integrated implementation, AHU controller 330 may be a software module configured for execution by a processor of BMS controller 366.

In some embodiments, AHU controller 330 receives information from BMS controller 366 (e.g., commands, setpoints, operating boundaries, etc.) and provides information to BMS controller 366 (e.g., temperature measurements, valve or actuator positions, operating statuses, diagnostics, etc.). For example, AHU controller 330 may provide BMS controller 366 with temperature measurements from temperature sensors 362-364, equipment on/off states, equipment operating capacities, and/or any other information that can be used by BMS controller 366 to monitor or control a variable state or condition within building zone 306.

Client device 368 may include one or more human-machine interfaces or client interfaces (e.g., graphical user interfaces, reporting interfaces, text-based computer interfaces, client-facing web services, web servers that provide pages to web clients, etc.) for controlling, viewing, or otherwise interacting with HVAC system 100, its subsystems, and/or devices. Client device 368 may be a computer workstation, a client terminal, a remote or local interface, or any other type of user interface device. Client device 368 may be a stationary terminal or a mobile device. For example, client device 368 may be a desktop computer, a computer server with a user interface, a laptop computer, a tablet, a smartphone, a PDA, or any other type of mobile or non-mobile device. Client device 368 may communicate with BMS controller 366 and/or AHU controller 330 via communications link 372.

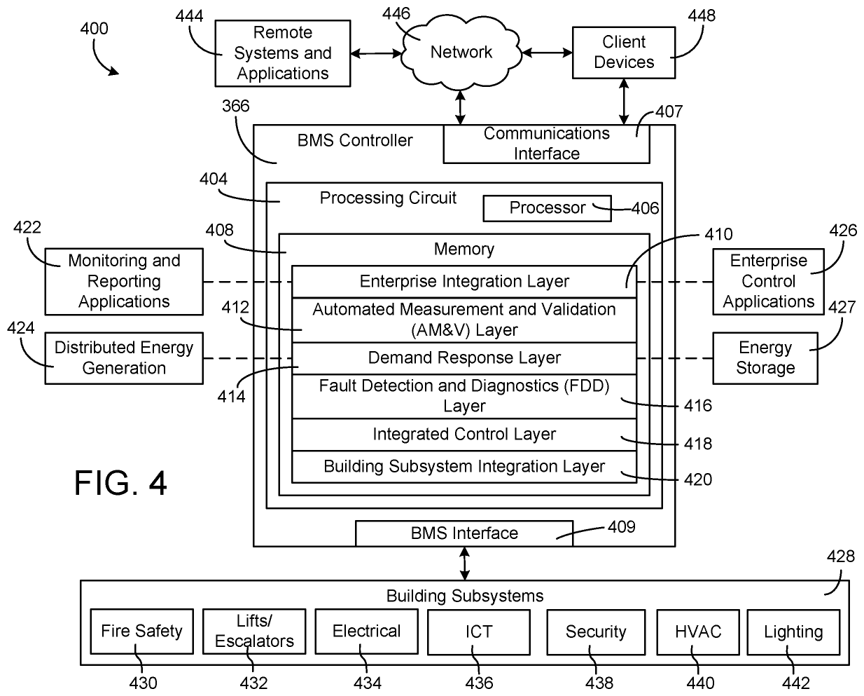

Referring now to FIG. 4, a block diagram of a building automation system (BMS) 400 is shown, according to some embodiments. BMS 400 may be implemented in building 10 to automatically monitor and control building functions. BMS 400 is shown to include BMS controller 366 and a plurality of building subsystems 428. Building subsystems 428 are shown to include a building electrical subsystem 434, an information communication technology (ICT) subsystem 436, a security subsystem 438, a HVAC subsystem 440, a lighting subsystem 442, a lift/escalators subsystem 432, and a fire safety subsystem 430. In embodiments, building subsystems 428 can include fewer, additional, or alternative subsystems. For example, building subsystems 428 may also or alternatively include a refrigeration subsystem, an advertising or signage subsystem, a cooking subsystem, a vending subsystem, a printer or copy service subsystem, or any other type of building subsystem that uses controllable equipment and/or sensors to monitor or control building 10. In some embodiments, building subsystems 428 include waterside system 200 and/or airside system 300, as described with reference to FIGS. 2-3.

Each of building subsystems 428 may include any number of devices, controllers, and connections for completing its individual functions and control activities. HVAC subsystem 440 may include many of the same components as HVAC system 100, as described with reference to FIGS. 1-3. For example, HVAC subsystem 440 may include a chiller, a boiler, any number of air handling units, economizers, field controllers, supervisory controllers, actuators, temperature sensors, and other devices for controlling the temperature, humidity, airflow, or other variable conditions within building 10. Lighting subsystem 442 may include any number of light fixtures, ballasts, lighting sensors, dimmers, or other devices configured to controllably adjust the amount of light provided to a building space. Security subsystem 438 may include occupancy sensors, video surveillance cameras, digital video recorders, video processing servers, intrusion detection devices, access control devices and servers, or other security-related devices.

Still referring to FIG. 4, BMS controller 366 is shown to include a communications interface 407 and a BMS interface 409. Interface 407 may facilitate communications between BMS controller 366 and external applications (e.g., monitoring and reporting applications 422, enterprise control applications 426, remote systems and applications 444, applications residing on client devices 448, etc.) for allowing user control, monitoring, and adjustment to BMS controller 366 and/or subsystems 428. Interface 407 may also facilitate communications between BMS controller 366 and client devices 448. BMS interface 409 may facilitate communications between BMS controller 366 and building subsystems 428 (e.g., HVAC, lighting security, lifts, power distribution, business, etc.).

Interfaces 407, 409 can be or include wired or wireless communications interfaces (e.g., jacks, antennas, transmitters, receivers, transceivers, wire terminals, etc.) for conducting data communications with building subsystems 428 or other external systems or devices. In embodiments, communications via interfaces 407, 409 may be direct (e.g., local wired or wireless communications) or via a communications network 446 (e.g., a WAN, the Internet, a cellular network, etc.). For example, interfaces 407, 409 can include an Ethernet card and port for sending and receiving data via an Ethernet-based communications link or network. In another example, interfaces 407, 409 can include a WiFi transceiver for communicating via a wireless communications network. In another example, one or both of interfaces 407, 409 may include cellular or mobile phone communications transceivers. In one embodiment, communications interface 407 is a power line communications interface and BMS interface 409 is an Ethernet interface. In other embodiments, both communications interface 407 and BMS interface 409 are Ethernet interfaces or are the same Ethernet interface.

Still referring to FIG. 4, BMS controller 366 is shown to include a processing circuit 404 including a processor 406 and memory 408. Processing circuit 404 may be communicably connected to BMS interface 409 and/or communications interface 407 such that processing circuit 404 and the components thereof can send and receive data via interfaces 407, 409. Processor 406 can be implemented as a general purpose processor, an application specific integrated circuit (ASIC), one or more field programmable gate arrays (FPGAs), a group of processing components, or other suitable electronic processing components.

Memory 408 (e.g., memory, memory unit, storage device, etc.) may include one or more devices (e.g., RAM, ROM, Flash memory, hard disk storage, etc.) for storing data and/or computer code for completing or facilitating the processes, layers and modules described in the present application. Memory 408 may be or include volatile memory or non-volatile memory. Memory 408 may include database components, object code components, script components, or any other type of information structure for supporting the activities and information structures described in the present application. According to an exemplary embodiment, memory 408 is communicably connected to processor 406 via processing circuit 404 and includes computer code for executing (e.g., by processing circuit 404 and/or processor 406) one or more processes described herein.

In some embodiments, BMS controller 366 is implemented within a single computer (e.g., one server, one housing, etc.). In other embodiments BMS controller 366 may be distributed across multiple servers or computers (e.g., that can exist in distributed locations). Further, while FIG. 4 shows applications 422 and 426 as existing outside of BMS controller 366, in some embodiments, applications 422 and 426 may be hosted within BMS controller 366 (e.g., within memory 408).

Still referring to FIG. 4, memory 408 is shown to include an enterprise integration layer 410, an automated measurement and validation (AM&V) layer 412, a demand response (DR) layer 414, a fault detection and diagnostics (FDD) layer 416, an integrated control layer 418, and a building subsystem integration later 420. Layers 410-420 may be configured to receive inputs from building subsystems 428 and other data sources, determine optimal control actions for building subsystems 428 based on the inputs, generate control signals based on the optimal control actions, and provide the generated control signals to building subsystems 428. The following paragraphs describe some of the general functions performed by each of layers 410-420 in BMS 400.

Enterprise integration layer 410 may be configured to serve clients or local applications with information and services to support a variety of enterprise-level applications. For example, enterprise control applications 426 may be configured to provide subsystem-spanning control to a graphical user interface (GUI) or to any number of enterprise-level business applications (e.g., accounting systems, user identification systems, etc.). Enterprise control applications 426 may also or alternatively be configured to provide configuration GUIs for configuring BMS controller 366. In yet other embodiments, enterprise control applications 426 can work with layers 410-420 to optimize building performance (e.g., efficiency, energy use, comfort, or safety) based on inputs received at interface 407 and/or BMS interface 409.

Building subsystem integration layer 420 may be configured to manage communications between BMS controller 366 and building subsystems 428. For example, building subsystem integration layer 420 may receive sensor data and input signals from building subsystems 428 and provide output data and control signals to building subsystems 428. Building subsystem integration layer 420 may also be configured to manage communications between building subsystems 428. Building subsystem integration layer 420 translate communications (e.g., sensor data, input signals, output signals, etc.) across a plurality of multi-vendor/multi-protocol systems.

Demand response layer 414 may be configured to optimize resource usage (e.g., electricity use, natural gas use, water use, etc.) and/or the monetary cost of such resource usage in response to satisfy the demand of building 10. The optimization may be based on time-of-use prices, curtailment signals, energy availability, or other data received from utility providers, distributed energy generation systems 424, from energy storage 427 (e.g., hot TES 242, cold TES 244, etc.), or from other sources. Demand response layer 414 may receive inputs from other layers of BMS controller 366 (e.g., building subsystem integration layer 420, integrated control layer 418, etc.). The inputs received from other layers may include environmental or sensor inputs such as temperature, carbon dioxide levels, relative humidity levels, air quality sensor outputs, occupancy sensor outputs, room schedules, and the like. The inputs may also include inputs such as electrical use (e.g., expressed in kWh), thermal load measurements, pricing information, projected pricing, smoothed pricing, curtailment signals from utilities, and the like.

According to an exemplary embodiment, demand response layer 414 includes control logic for responding to the data and signals it receives. These responses can include communicating with the control algorithms in integrated control layer 418, changing control strategies, changing setpoints, or activating/deactivating building equipment or subsystems in a controlled manner. Demand response layer 414 may also include control logic configured to determine when to utilize stored energy. For example, demand response layer 414 may determine to begin using energy from energy storage 427 just prior to the beginning of a peak use hour.

In some embodiments, demand response layer 414 includes a control module configured to actively initiate control actions (e.g., automatically changing setpoints) which minimize energy costs based on one or more inputs representative of or based on demand (e.g., price, a curtailment signal, a demand level, etc.). In some embodiments, demand response layer 414 uses equipment models to determine an optimal set of control actions. The equipment models may include, for example, thermodynamic models describing the inputs, outputs, and/or functions performed by sets of building equipment. Equipment models may represent collections of building equipment (e.g., subplants, chiller arrays, etc.) or individual devices (e.g., individual chillers, heaters, pumps, etc.).

Demand response layer 414 may further include or draw upon one or more demand response policy definitions (e.g., databases, XML files, etc.). The policy definitions may be edited or adjusted by a user (e.g., via a graphical user interface) so that the control actions initiated in response to demand inputs may be tailored for the user's application, desired comfort level, particular building equipment, or based on other concerns. For example, the demand response policy definitions can specify which equipment may be turned on or off in response to particular demand inputs, how long a system or piece of equipment should be turned off, what setpoints can be changed, what the allowable set point adjustment range is, how long to hold a high demand setpoint before returning to a normally scheduled setpoint, how close to approach capacity limits, which equipment modes to utilize, the energy transfer rates (e.g., the maximum rate, an alarm rate, other rate boundary information, etc.) into and out of energy storage devices (e.g., thermal storage tanks, battery banks, etc.), and when to dispatch on-site generation of energy (e.g., via fuel cells, a motor generator set, etc.).

Integrated control layer 418 may be configured to use the data input or output of building subsystem integration layer 420 and/or demand response later 414 to make control decisions. Due to the subsystem integration provided by building subsystem integration layer 420, integrated control layer 418 can integrate control activities of the subsystems 428 such that the subsystems 428 behave as a single integrated super-system. In an exemplary embodiment, integrated control layer 418 includes control logic that uses inputs and outputs from a plurality of building subsystems to provide greater comfort and energy savings relative to the comfort and energy savings that separate subsystems could provide alone. For example, integrated control layer 418 may be configured to use an input from a first subsystem to make an energy-saving control decision for a second subsystem. Results of these decisions can be communicated back to building subsystem integration layer 420.

Integrated control layer 418 is shown to be logically below demand response layer 414. Integrated control layer 418 may be configured to enhance the effectiveness of demand response layer 414 by enabling building subsystems 428 and their respective control loops to be controlled in coordination with demand response layer 414. This configuration may advantageously reduce disruptive demand response behavior relative to conventional systems. For example, integrated control layer 418 may be configured to assure that a demand response-driven upward adjustment to the setpoint for chilled water temperature (or another component that directly or indirectly affects temperature) does not result in an increase in fan energy (or other energy used to cool a space) that would result in greater total building energy use than was saved at the chiller.

Integrated control layer 418 may be configured to provide feedback to demand response layer 414 so that demand response layer 414 checks that constraints (e.g., temperature, lighting levels, etc.) are properly maintained even while demanded load shedding is in progress. The constraints may also include setpoint or sensed boundaries relating to safety, equipment operating limits and performance, comfort, fire codes, electrical codes, energy codes, and the like. Integrated control layer 418 is also logically below fault detection and diagnostics layer 416 and automated measurement and validation layer 412. Integrated control layer 418 may be configured to provide calculated inputs (e.g., aggregations) to these higher levels based on outputs from more than one building subsystem.

Automated measurement and validation (AM&V) layer 412 may be configured to verify that control strategies commanded by integrated control layer 418 or demand response layer 414 are working properly (e.g., using data aggregated by AM&V layer 412, integrated control layer 418, building subsystem integration layer 420, FDD layer 416, or otherwise). The calculations made by AM&V layer 412 may be based on building system energy models and/or equipment models for individual BMS devices or subsystems. For example, AM&V layer 412 may compare a model-predicted output with an actual output from building subsystems 428 to determine an accuracy of the model.

Fault detection and diagnostics (FDD) layer 416 may be configured to provide on-going fault detection for building subsystems 428, building subsystem devices (i.e., building equipment), and control algorithms used by demand response layer 414 and integrated control layer 418. FDD layer 416 may receive data inputs from integrated control layer 418, directly from one or more building subsystems or devices, or from another data source. FDD layer 416 may automatically diagnose and respond to detected faults. The responses to detected or diagnosed faults may include providing an alert message to a user, a maintenance scheduling system, or a control algorithm configured to attempt to repair the fault or to work-around the fault.

FDD layer 416 may be configured to output a specific identification of the faulty component or cause of the fault (e.g., loose damper linkage) using detailed subsystem inputs available at building subsystem integration layer 420. In other exemplary embodiments, FDD layer 416 is configured to provide “fault” events to integrated control layer 418 which executes control strategies and policies in response to the received fault events. According to an exemplary embodiment, FDD layer 416 (or a policy executed by an integrated control engine or business rules engine) may shut-down systems or direct control activities around faulty devices or systems to reduce energy waste, extend equipment life, or assure proper control response.

FDD layer 416 may be configured to store or access a variety of different system data stores (or data points for live data). FDD layer 416 may use some content of the data stores to identify faults at the equipment level (e.g., specific chiller, specific AHU, specific terminal unit, etc.) and other content to identify faults at component or subsystem levels. For example, building subsystems 428 may generate temporal (i.e., time-series) data indicating the performance of BMS 400 and the components thereof. The data generated by building subsystems 428 may include measured or calculated values that exhibit statistical characteristics and provide information about how the corresponding system or process (e.g., a temperature control process, a flow control process, etc.) is performing in terms of error from its setpoint. These processes can be examined by FDD layer 416 to expose when the system begins to degrade in performance and alert a user to repair the fault before it becomes more severe.

Facility with Smart Features

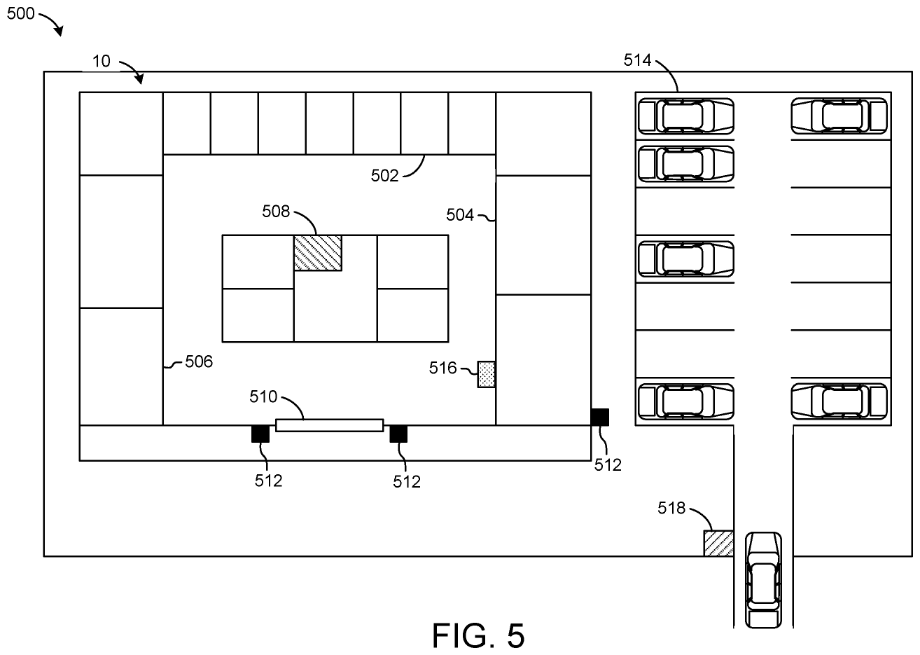

Referring now to FIG. 5, a diagram of a site 500 that includes building 10 is shown, according to some embodiments. In some embodiments, the building 10 is a hospital and includes a number of different rooms (i.e., spaces), such as room 502, room 504, and room 506. Each of rooms 502-506 may be configured a different type of room and/or may be configured for a unique purpose. For example, in a hospital facility, room 502 may represent a patient room, room 504 may represent a surgery room, and room 506 may represent a meeting room. It will be appreciated that building 10 may include any number of rooms, even beyond the number of rooms shown in FIG. 5, and each room may be any type of room depending on the configuration of building 10. For example, if building 10 is an office building rather than a hospital, then rooms 502-506 may consist of offices, meeting rooms, break rooms, etc. Any combination of rooms or spaces within building 10 is considered herein.

In some embodiments, building 10 may include other spaces, devices, or components in addition to rooms 502-506. In such embodiments, building 10 may include an elevator 508, although in some embodiments, elevator 508 may be replaced with a stair case, and escalator, or other similar component. Likewise, building 10 can include any of the equipment described above with respect to FIGS. 1-4. Building 10 is also shown to include an entryway 510, which may be a main entrance to building 10. It will be appreciated that building 10 may also include other entry and exit points (e.g., additional doorways, stairs, hallways, etc.) that are not shown in FIG. 5.

Monitoring entryway 510 are one or more security devices 512. Security devices 512 can include any device or component of a security system, such as security subsystem 438 described above, including but not limited to video surveillance cameras, motion sensors, security lights, audio sensors, etc. In particular, security devices 512 may include at least video cameras configured to capture video and/or image data from one or more sides of building 10. In the example shown, security devices 512 may capture video of persons entering building 10. Building 10 may additionally include any number of other security devices 512 positioned around the interior or exterior of the building. For example, security devices 512 can include cameras configured to monitor a parking structure 514 (e.g., a parking lot, a parking garage, etc.).

Building 10 may also include a number of access terminals 516 positioned in common areas, such as in hallways. Access terminals 516 may include a memory (e.g., RAM, ROM, Flash memory, hard disk storage, etc.), a processor (e.g., a general purpose processor, an application specific integrated circuit (ASIC), one or more field programmable gate arrays (FPGAs), a group of processing components, or other suitable electronic processing components), and a user interface (e.g., a touch screen) for displaying information regarding building 10. In some embodiments, access terminals 516 may display a 2-dimensional (2D) floor map or a 3-dimensional (3D) model of building 10, to aid an occupant in navigation. For example, an occupant of building 10 may interact with access terminals 516 to determine their current location in building 10, or to determine an optimal route for navigating to another part of building 10 (e.g., a particular room). Access terminals 516 may also allow users to view information such as a staff directory, occupant/room directory, etc. In some embodiments, such as in a hospital setting, access terminals 516 may allow patients to check-in or check-out for appointments. It will be appreciated that access terminals 516 may also be configured to provide any of the additional smart building control functionality described herein.

In some embodiments, site 500 also includes an access control point 518, configured to control access into site 500. In some such embodiments, access control point 518 controls access to parking structure 514. Access control point 518 may be an automated or semi-automated system that includes components such as a user interface (e.g., a touch screen, a screen and keypad, etc.), barrier gates, an intercom, card and/or code readers, radio-frequency (RF) readers, etc. For example, access control point 518 may include a barrier that prevents access to parking structure 514 until an access code or token, a ticket, a payment, etc., is presented to a user interface. In this example, a user entering parking structure 514 may request a ticket that records the time that the user enters parking structure 514. The user may then present the ticket to access control point 518 when exiting parking structure 514 to raise the barrier. In some embodiments, the user may be required to make a payment to enter or exit parking structure 514. It will be appreciated that access control point 518 may also include other types of access control systems and devices not described herein.

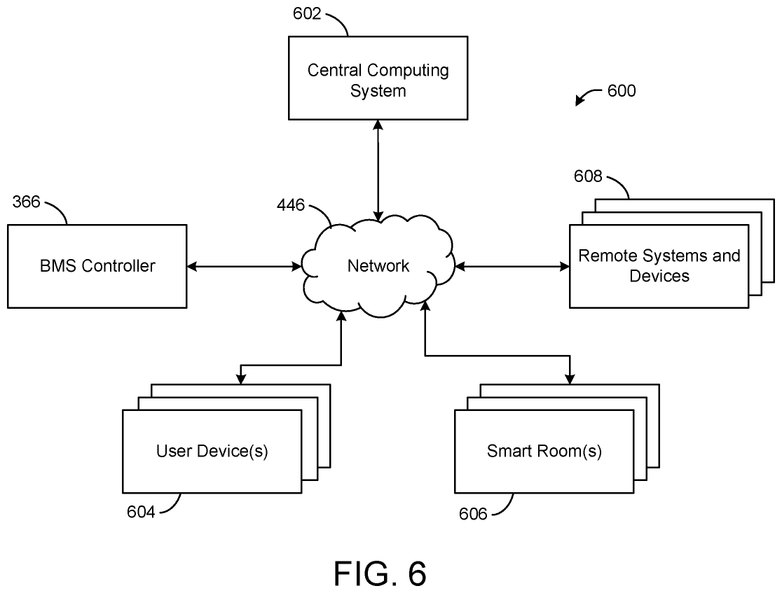

Referring now to FIG. 6, a block diagram of a control system 600 for a smart building (e.g., building 10) is shown, according to some embodiments. As described briefly above, system 600 may encompass any number of systems, subsystems, and devices that can be incorporated into building 10, thereby monitoring and controlling all of the systems in a building in unison. System 600 advantageously provides more robust and centralized control over all of the components of building 10, allowing for greater functionality and increase automation.

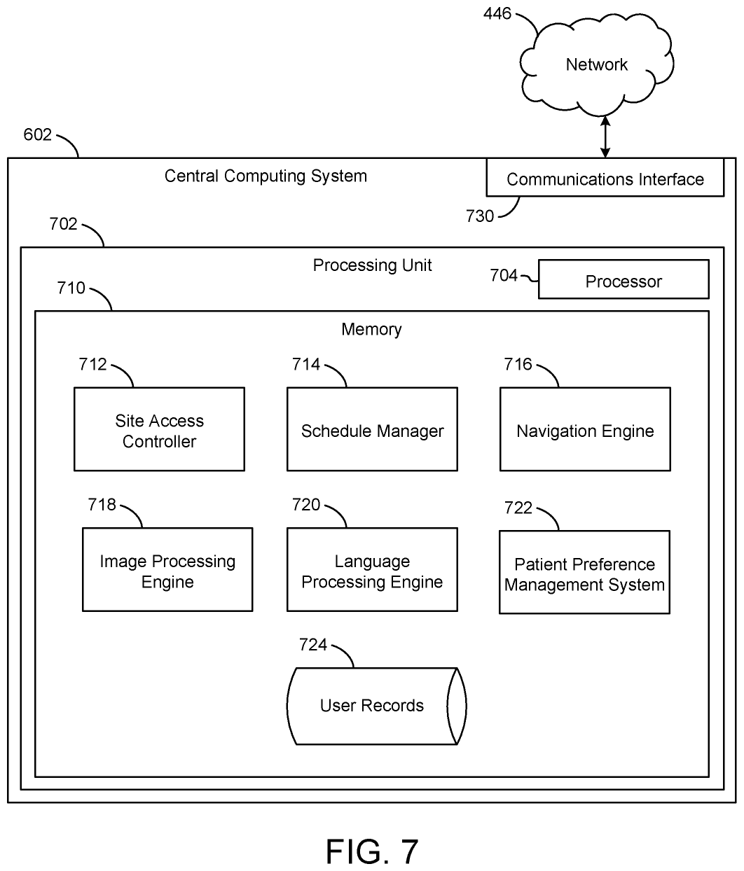

System 600 is shown to include a central computing system 602 (e.g., a server) configured to receive, process, store, and/or transmit data or signals from/to the various other components of system 600. In particular, central computing system 602 may be configured to receive and transmit data via network 446, which may be any suitable wired or wireless network as described above, and may be configured to process received data to determine control decisions and enable smart features in building 10. In some embodiments, central computing system 602 communicates (e.g., receives and transmits data from/to) with BMS controller 366, as described in detail above with respect to FIG. 3. In this manner, central computing system 602 may monitor and/or process BMS or building device data (e.g., sensor readings, device parameters, etc.), and may also transmit control signals to cause BMS controller 366 to control building devices. Central computing system 602 is described in greater detail below, with respect to FIG. 7.

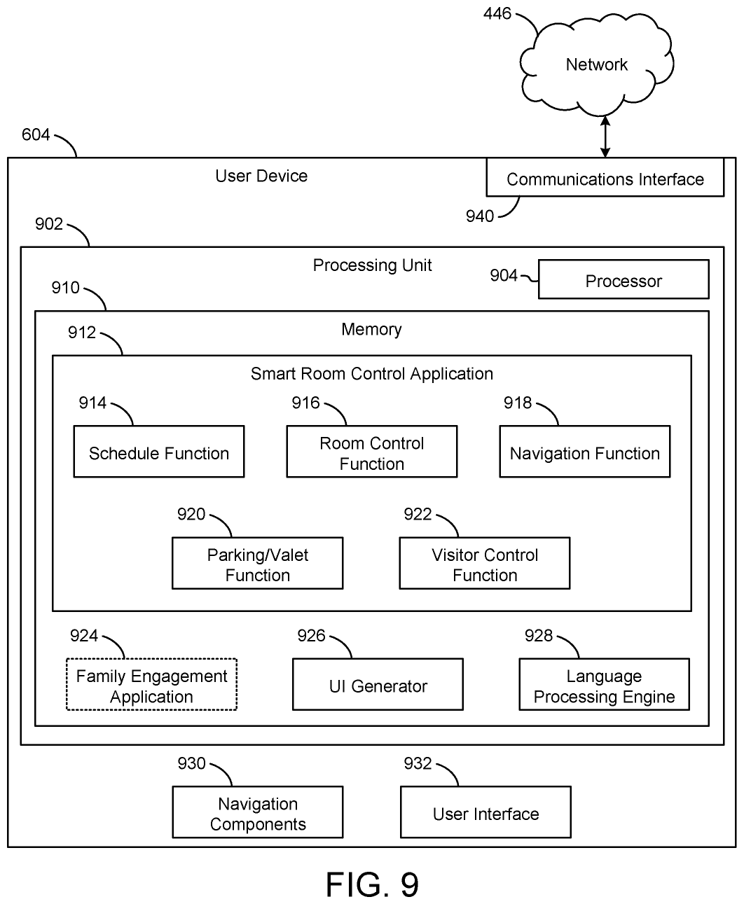

System 600 also includes user device(s) 604. Each of user device(s) 604 may be a computing device including a memory (e.g., RAM, ROM, Flash memory, hard disk storage, etc.), a processor (e.g., a general purpose processor, an application specific integrated circuit (ASIC), one or more field programmable gate arrays (FPGAs), a group of processing components, or other suitable electronic processing components), and a user interface (e.g., a touch screen), allowing a user to interact with system 600. User device(s) 604 can include, for example, mobile phones, electronic tablets, laptops, desktop computers, workstations, vehicle dashboards, and other types of electronic devices. More generally, user device(s) 604 may include any electronic device that allows a user to interact with system 600 (e.g., through a user interface). In some embodiments, user device(s) 604 may be connected to network 446 via an intranet or via the Internet, either via a wired connection or a wireless connection. User device(s) 604 are described in greater detail below, with respect to FIG. 9.

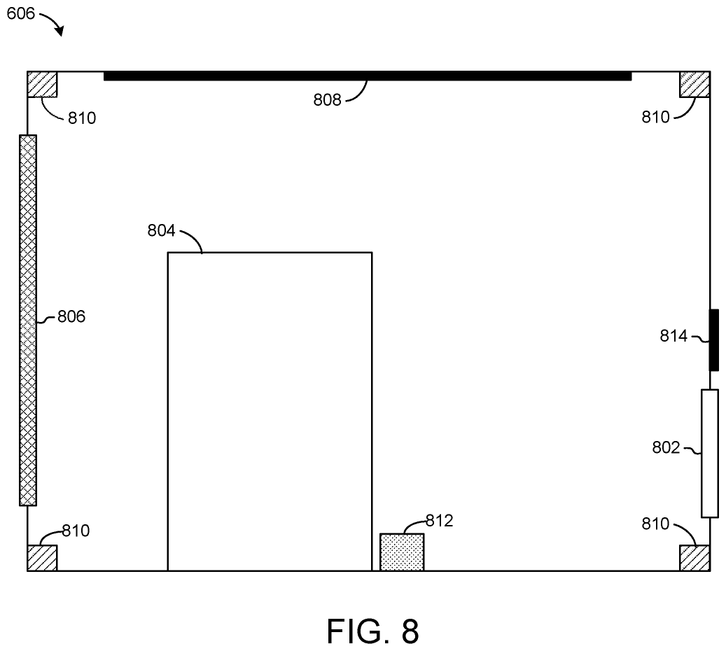

System 600 is also shown to include smart room(s) 606, also referred to as “sentient” rooms. Smart room(s) 606, such as rooms 502-506 described above, may be any type of space within building 10 that includes one or more smart features or devices. For example, a smart room 606 can include any of building subsystems 428 and/or any components of building subsystems 428, as described above. Smart room(s) 606 may actively monitor occupancy and occupant comfort to modify operating constraints, such as temperature, pressure, humidity, lighting, noise level, etc. In a hospital, for example, a smart patient room may be configured to maintain temperature, pressure, and humidity to ensure patient comfort, mitigate infection risk, and improve healing times. Additionally, smart room(s) 606 may interact with occupants, such as by receiving voice commands via an audio sensor, to adjust room parameters (e.g., raise the temperature, turn on a television, play music, etc.), thereby increasing automation and reducing manual user input. Smart room(s) 606 are described in greater detail below with respect to FIG. 8.

Still referring to FIG. 6, system 600 may also include remote systems and devices 608. Remote systems and devices 608 may include any system, subsystem, or device that is not included in a BMS system for building 10, but that may still be implemented in building 10 and/or utilized by the various other components of system 600. For example, remote systems and devices 608 may include a central server or other computing device for a building, or may include a computing device for various functions within an organization. In this example, remote systems and devices 608 may include a scheduling system that tracks and modifies user schedules (e.g., patient appointments, meetings, etc.). Remote systems and devices 608 may also include systems that provide outside information, such as weather, traffic, news, etc. In some embodiments, such as in a healthcare facility, remote systems and devices 608 can include a computing system for a pharmacy, which may be separate from other healthcare systems. Accordingly, central computing system 602 and/or user devices 604 may be configured to retrieve and/or receive pharmacy information for a user, as described in greater detail below. It will be appreciated that any remote systems or devices that is not described above with respect to FIGS. 1-4 is considered herein, and may be incorporated into remote systems and devices 608.

The network 446 of system 600 of FIG. 6 is shown as a cloud based computing system. In some embodiments, the network 446 can include a local server system, a remote server system, a distributed control and data storage system, or any combination of network systems. In some embodiments, various data discussed herein may be processed at (e.g., processed using models executed at) the network 446 or other off-premises computing system/device or group of systems/devices, an edge or other on-premises system/device or group of systems/devices (e.g., the central computing system 602, the user device(s) 604, the smart room(s) 606, the remote system and devices 608, and/or the BMS controller 366), or a hybrid thereof in which some processing occurs off-premises and some occurs on-premises. In some example implementations, the data may be processed using systems and/or methods such as those described in U.S. patent application Ser. No. 17/710,458 filed Mar. 31, 2022, which is incorporated herein by reference in its entirety. In some embodiments, aspects of the system 600 may be incorporated into the network 446 or other network system(s). Additionally, in some embodiments, various data discussed herein may be stored in, retrieved from, or processed in the context of digital twins. In some such embodiments, the digital twins may be provided within an infrastructure such as those described in U.S. patent application Ser. No. 17/134,661 filed Dec. 28, 2020, 63/289,499 filed Dec. 14, 2021, and Ser. No. 17/537,046 filed Nov. 29, 2021, the entireties of each of which are incorporated herein by reference.

Referring now to FIG. 7, a block diagram of central computing system 602 included in system 600 is shown, according to some embodiments. As described briefly above, central computing system 602 may be any suitable computing device or system, such as a server, a desktop or laptop computer, etc. In some embodiments, central computing system 602 is hosted locally to building 10 (e.g., central computing system 602 is installed in building 10). In other embodiments, central computing system 602 is a remote system, such as a server host off-site from building 10 (e.g., a cloud server). In any case, central computing system 602 may be configured to perform a variety of task to enable various “smart” functions within building 10, including at least the various functions described below.

Central computing system 602 is shown to include a processing circuit 702, which includes a processor 704 and memory 710. It will be appreciated that these components can be implemented using a variety of different types and quantities of processors and memory. For example, processor 704 can be a general purpose processor, an application specific integrated circuit (ASIC), one or more field programmable gate arrays (FPGAs), a group of processing components, or other suitable electronic processing components. Processor 704 can be communicatively coupled to memory 710. While processing circuit 702 is shown as including one processor 704 and one memory 710, it should be understood that, as discussed herein, a processing circuit and/or memory may be implemented using multiple processors and/or memories in various embodiments. All such implementations are contemplated within the scope of the present disclosure.

Memory 710 can include one or more devices (e.g., memory units, memory devices, storage devices, etc.) for storing data and/or computer code for completing and/or facilitating the various processes described in the present disclosure. Memory 710 can include random access memory (RAM), read-only memory (ROM), hard drive storage, temporary storage, non-volatile memory, flash memory, optical memory, or any other suitable memory for storing software objects and/or computer instructions. Memory 710 can include database components, object code components, script components, or any other type of information structure for supporting the various activities and information structures described in the present disclosure. Memory 710 can be communicably connected to processor 704 via processing circuit 702 and can include computer code for executing (e.g., by processor 704) one or more processes described herein.

Memory 710 is shown to include a site access controller 712 configured to manage site and/or building access (e.g., entry and exit). In some embodiments, site access controller 712 communicates with a remote (i.e., external) access control system (e.g., an access control system not included in system 600). In such embodiments, the access control system and/or one or more access control devices can be included in security subsystem 438. Access control devices can include, for example, card or badge readers, electronic locks, RFID tag readers, finger print or other biometric scanners, etc., which operate by monitoring and limiting occupant access to various areas in a building. In one example, access control for a room (e.g., room 502) of building 10 may include a card or badge reader and an electronic lock, where a door to the room remains locked until a user having a suitable security clearance or access level presents a card or badge to the reader.

In some embodiments, site access controller 712 may also monitor the location of a plurality of users (e.g., occupants of building 10). For example, site access controller 712 may track users as they enter and exit spaces within building 10 (e.g., when then swipe a badge to enter a room) to determine the user's location. In some embodiments, site access controller 712 performs check-in and check-out procedures for occupants as they enter/exit building 10. For example, in a hospital, site access controller 712 may check-in a person entering building 10 for an upcoming appointment. A check-in procedure may include identifying the occupant, retrieving occupant information, and updating a database to indicate that the occupant is within site 500 and/or checked in. In this regard, site access controller 712 may communicate with various other components of memory 712 to perform check-in or check-out procedures, as described in greater detail below.

Memory 710 is also shown to include a schedule manager 714 configured to manage user (i.e., occupant) schedules, as well as to check-in and check-out building occupants (e.g., in cooperation with site access controller 712). Schedule manager 714 may be configured to maintain a database (e.g., user records 724) that includes schedules for one or more occupants of building 10, and in some cases includes schedules associated with one or more spaces of building 10. For example, schedule manager 714 may manage (e.g., track and update) reservation times for meeting rooms. In another example, schedule manager 714 may monitor schedules for various operating rooms of a hospital to ensure that scheduled surgeries do not overlap.

In some embodiments, schedule manager 714 may maintain schedules for both regular occupants of building 10 such as doctors, nurses, and other staff in a hospital setting, as well as occasional occupants of building 10 such as patients. For example, schedule manager 714 may manage appointment times for patients, and in some cases may even generate and transmit (e.g., via network 446) notifications to patients regarding past, upcoming, or missed appointments. In some embodiments, schedule manager 714 communicates with site access controller 712 to check-in or check-out users as they enter or exit building 10. For example, when a new person (e.g., a patient) enters building 10 and is identified by site access controller 712 and/or other components of memory 710, schedule manager 714 may determine whether the identified person has an upcoming appointment. Assuming the identified person has a scheduled appointment, site access controller 712 may check-in the identified person for the appointment. If the identified person does not have an appointment, site access controller 712 may notify building staff (e.g., a receptionist) and/or may generate and transmit a notification to the identified person's mobile device requesting the identified person check-in.

Memory 710 is shown to include a navigation engine 716 configured to generate internal and external navigation data for building 10, as well as to manage parking in one or more parking structures (e.g., parking structure 514) on site 500. Navigation engine 716 may receive navigation requests from users (e.g., via user devices 604) that generally include a starting location (e.g., the user's current location) and a destination (e.g., building 10). In some embodiments, navigation engine 716 may identify a starting location based on the requesting user's current location, as determined by GPS data or other location data received with the navigation request. Navigation engine 716 may generate a route from the starting location to the destination according to one or more parameters, and may subsequently transmit the generated route to the user's device. In some embodiments, navigation engine 716 may communicate with a remote or third-party system when generating the route. For example, navigation engine 716 may interface with Google Maps® or another similar navigation service to generate routes. In such embodiments, navigation engine 716 may identify a requesting user's current location and may transmit the identified location and the destination location to the remote service for processing.

In some embodiments, navigation engine 716 may automatically generate a route for a user based on a determination that the user has an upcoming appointment. For example, schedule manager 714 may determine that a user (e.g., a patient) has an upcoming appointment and may automatically query navigation engine 716 to generate a route from the user's location (e.g., current or predetermined, such as a home address) to building 10. In some embodiments, navigation engine 716 may also prompt the user to reserve parking at the destination. In such embodiments, navigation engine 716 can update the destination address for the requested navigation to the particular parking spot or parking structure reserved by the user. In some embodiments, navigation engine 716 may also generate and transmit, to the user's device, a token or a code that allows the user entry to the parking structure. In some embodiments, navigation engine 716 also communicates with a remote and/or third-party payment service to collect a payment for reservation of the parking space.

Memory 710 is shown to include an image processing engine 718 configured to process image data and, in some cases, to identify occupants based on the processed image data. In particular, image processing engine 718 may receive image data from one or more security devices 512 positioned around building 10, and may process the received data to identify occupants or other features. For example, security devices 512 positioned near an entrance to building 10 (e.g., as shown in FIG. 5) may capture image data (e.g., video) of persons entering and/or exiting building 10. Image processing engine 718 may be configured to analyze this image data to identify persons entering and/or exiting the building, such as by searching for matching images in a user database (e.g., user records 724). In some embodiments, image processing engine 718 includes a neural network or other artificial intelligence for identifying occupants of building 10. For example, image processing engine 718 may include a convolution neural network (CNN) for image recognition.

Memory 710 is shown to include a language processing engine 720 configured to process spoken (i.e., natural) language to generate commands. More generally, language processing engine 720 may include natural language processing (NLP), or may communicate with a remote and/or third-party NLP service, to process spoken commands received from occupants of a building. In some embodiments, audio data is received from one or more audio devices (e.g., microphones, smart speakers, mobile phones, etc.) positioned throughout building 10. These audio devices may be configured to continuously and/or occasionally monitor and/or record sounds (e.g., human speech), which is transmitted to language processing engine 720 via network 446 for processing. Language processing engine 720 may include a neural network or artificial intelligence that can convert the audio data into text-based data or other similar data that can be interpreted by central computing system 602. For example, language processing engine 720 can convert the audio data into commands that can control various functions of system 600, as described in greater detail below.

Memory 710 is also shown to include a patient preference management system (PPMS) 722 configured to determine, record, store, and/or retrieve various patient preferences. Patient preferences may include, for example, preferred temperature, pressure, and humidity levels in a room (e.g., one of smart rooms 606), preferred lighting temperatures and intensities, preferred meal times, favorite movies or television shows, favorite musicians and bands, a patient's sleep schedule, etc., along with any of the other patient or user preferences described herein. In some embodiments, patient preferences are entered manually, such as by a patient or facility (e.g., hospital) staff when the patient is checking-in or registering. In some embodiments, PPMS 722 is configured to automatically determine patient preferences by recording room and/or schedule parameters over time (e.g., temperature, pressure, and humidity levels in the patient's room) and/or by analyzing the patient's social media accounts, bank and/or credit card purchases, search history, etc. For example, PPMS 722 may receive preference information for a patient from a remote and/or third-party (e.g., Google®, Facebook®, etc.).

In some embodiments, the PPMS 722 is configured to determine a state-of-mind (SoM) of the patient. SOM can include a value or score determined based on inputs and provides an estimate of how healthy the patient is feeling mentally. For example, is the patient happy or sad, in pain or comfortable, lonely or feeling supported. In some embodiments, the SOM can be represented by a grid, a quadrant chart, a string, a single value, or another parameter. The PPMS 722 can receive inputs from sensors and devices within a smart room 606. For example, devices and sensors can include a temperature sensor, a pressure sensor, a humidity sensor, a camera with an optical analysis system, a bed weight sensor, an occupancy sensor, an air quality sensor (e.g., oxygen percentage sensor, contaminant ppm sensor, carbon dioxide sensor, etc.), a nurse call button, a thermostat, a wall panel touch screen, etc. In some embodiments, SOM is determined at the edge in a computing system within the smart room 606, adjacent the smart room 606, or at a nearby nurse station or room. Providing on-premises determination of SOM at the edge can reduce the computational load and required data transmission to or over the network 446. Additionally, the separation of SOM determination at the edge and BMS controls via the BMS 366, or central computing system 602 may provide patient confidentiality advantages. For example, electronic medical records (EMR) may be accessed via a secure server connected to the network to retrieve information relative to SOM of the patient (e.g., historical SOM information, prevalence of depression in family history, past medical emergencies or experiences, etc.). The EMR information can be used to train a learning model in the form of a SOM model or SOM machine learning policy in the offsite and/or secure server. Once the SOM machine learning policy is trained, then the SOM machine learning policy can be uploaded to the edge devices of the smart room 606 so that the EMR information can be utilized without actually transferring the confidential information stored in the EMR to any devices on-premises or to any section of the network 446 that is not secured. In some embodiments, the SOM machine learning policy receives a data set of historical environmental changes and correlated patient reactions during training. For example, patients with a particular demographic, gender, weight, height, psychiatric disposition, etc. may present identifiable correlations to environmental factors that could be useful in building and training the SOM machine learning policy. The SOM machine learning policy can build patient profiles based on the data set, and the SOM machine learning policy can then be used to associate the patient in the smart room 606 with a defined patient profile. The SOM machine learning policy can then utilize the patient profile to affect the determination of the SOM score and thereby the control commands sent to building devices (e.g., room device(s), floor device(s), local area device(s), etc.) and the HVAC system to improve the SOM score of the patient while maintaining TPH within the compliance standards.

In some embodiments, the SOM machine learning policy receives inputs including EMR information, other patient historical information, patient defined preferences (e.g., as discussed above), a number of visitors (e.g., a occupancy sensor or optical device can count visitors), a number of visitors per hour, a number of visitors during selected time periods (e.g., during breakfast, lunch, dinner, during a favorite TV show time, etc.), a facial recognition system (e.g., determine if the patient is frowning, grimacing, smiling, laughing, shivering, removing blankets, etc.), a body language recognition system (e.g., is the patient standing, pacing, constantly moving, staying perfectly still, etc.), a thermostat (e.g., is the patient constantly changing the thermostat temperature, a blower speed, etc.), a nurse call system (e.g., count the number of nurse call made, a number of nurse calls made per hour, etc.), a pain management system (e.g., monitor the patients reported pain, is the patient requesting more pain medication, etc.), and/or other inputs as desired.

The SOM machine learning policy receives the inputs and determines a SOM score. As discussed above, the SOM score can be a numeric value, a chart output, a letter grade, etc. In some embodiment, the patient interacts directly with the SOM machine learning policy by entering a patient entered SOM score. The patient entries can be used in a reinforcement learning scheme to update the SOM machine learning policy and drive future accuracy of the SOM score. The SOM score can then be used by the smart room 606 for operation of HVAC systems, room controls (e.g., lighting, blinds, entertainment systems, etc.), and hospital staff system (e.g., nurse call system, etc.) to provide a satisfying patient experience and improve the SOM score of the patient.

In some embodiments, PPMS 722 stores and/or retrieves patient preferences from user records 724, as described in greater detail below. In some embodiments, such as when central computing system 602 is implemented in a hospital or other healthcare facility, PPMS 722 may be optionally included in central computing system 602 as shown. In other embodiments, PPMS 722 may be at least partially implemented by a remote system (e.g., remote systems and device 608) or may not be included in central computing system 602 (e.g., when central computing system 602 is not utilized in a healthcare facility). It will be appreciated that all such implementations of PPMS 722 are contemplated herein.

Memory 710 is also shown to include user records 724, as briefly mentioned above. User records 724 may be a database, or may be included in a database, and may include records regarding any of the occupants or users of building 10. In some embodiments, user records 724 include employee records and/or customer or patient records. User records 724 may include personal information such as a name, address, phone number, email, social media account information, historical data, usage data, preferences, user inputted information, and any other information pertaining to an occupant of building 10. In some embodiments, user records 724 also include a user's schedule, such as an appointment schedule for a patient or a work schedule for an employee. It will be appreciated that any other information relating to an occupant or user of building 10 may also be included in user records 724.

Still referring to FIG. 7, central computing system 602 is also shown to include a communications interface 730. Communications interface 730 may facilitate communications between central computing system 602 and any of the other components of system 600. For example, communications interface 730 may allow central computing system 602 to receive data from user devices 604 via network 446, and to transfer data or commands to BMS controller 366. Accordingly, communications interface 730 can be or can include a wired or wireless communications interface (e.g., jacks, antennas, transmitters, receivers, transceivers, wire terminals, etc.) for conducting data communications.

In various embodiments, communications via communications interface 730 may be direct (e.g., local wired or wireless communications) or via network 446 (e.g., a WAN, the Internet, a cellular network, etc.). For example, communications interface 730 can include an Ethernet card and port for sending and receiving data via an Ethernet-based communications link or network. In another example, communications interface 730 can include a WiFi transceiver for communicating via a wireless communications network. In another example, communications interface 730 may include cellular or mobile phone communications transceivers.

Referring now to FIG. 8, a diagram of a smart room 606 (i.e., sentient room) located in building 10 is shown, according to some embodiments. Smart room 606 may include a number of components that include or enable “smart” features that enhance occupant comfort or otherwise improve the overall experience of occupants of smart room 606. In this regard, smart room 606 may include components that automate one or more processes that are typically handled manually in non-smart rooms. Additionally, smart room 606 and/or the components of smart room 606 may adapt to and/or learn an occupant's preferences, such as temperature, humidity, noise level, etc. In a hospital setting, smart room 606 may improve patient comfort and, in some cases, may reduce recovery time.

Smart room 606 is shown to include an entryway 802, which may be a manual or automatic door that secures the entrance to smart room 606, such as via an electronic lock. In this case, entryway 802 may be a component of an access control system for building 10, thereby controlling entry to and exit from smart room 606. In some embodiments, smart room 606 includes a bed 804 or other furniture that may include one or more motors, actuators, sensors, etc., for positioning a user and/or improving user comfort. For example, in a hospital, bed 804 may include a plurality of motors and a control unit for raising and/or lower portions of bed 804 to improve user comfort.

Smart room 606 also includes blinds 806, which may cover an interior or exterior window. Blinds 806 may be configured to prevent all, some, or very little light from entering smart room. Accordingly, blinds 806 may also include one or more motors or actuators to open/close blinds 806, thereby controlling the amount of outside light that can enter smart room 606. In some embodiments, smart room 606 includes a display 808, such as a television screen, a computer monitor, etc. In some such embodiments, display 808 is a “smart” wall that acts as a display screen, presenting information to a user. In such embodiments, display 808 may span across the majority of one or more walls of smart room 606.

Smart room 606 can also include one or more sensor arrays 810. Sensor arrays 810 can include a variety of sensors for measuring and/or recording parameters of smart room 606. In some embodiments, sensor arrays 810 include temperature, pressure, and humidity sensors, light level sensors, motion sensors, visible light and/or infrared cameras, microphones, air quality sensors, oxygen sensors, and any other suitable sensors. In this regard, sensor arrays 810 can monitor parameters such as temperature, pressure, and humidity of smart room 606, and the ambient light level in smart room 606, as well as monitoring parameters of occupants of smart room 606 such as body temperature, heartrate, position, etc. Such data may be used (e.g., by central computing system 602) to determine a comfort and/or stress level of one or more occupants of smart room 606.

In a hospital setting, smart room 606 may also be configured to detect the presence of a physician, nurse, or other hospital staff, and may subsequently initiate one or more response processes. In some embodiments, hospital staff may be detected via facial recognition as they enter smart room 606, or sensor arrays 810 can include other devices for tracking hospital staff. In some such embodiments, a real-time location system (RTLS) may be incorporated into smart room 606 and/or sensor arrays 810 for tracking corresponding RTLS tags carried by hospital staff. For example, a physician may carry an RFID enabled badge or a phone (e.g., with GPS and/or WiFi tracking) that can be detected as the physician enters smart room 606.

In some embodiments, responsive to detecting that a particular occupant (e.g., a physician) has entered smart room 606, a notification or alert can be transmitted to a remote device. As an example, a patient's family members may submit a request for live updates from a physician during a schedule appointment or procedure. When the attending physician is detected in smart room 606, a notification (e.g., a text, an email, a push notification, etc.) can be transmitted to a user device of the family member(s). In some embodiments, the notification can include a secure video conference link, allowing the family member to video conference with the patient and/or physician to discuss the patient's status, care plan, etc. In some embodiments, the video conference can be presented via display 808. In some embodiments, hospital staff may only be tracked during a predefined timeframe, such as during a schedule appointment or during certain hours (e.g., from 9 am to 5 pm) to avoid transmitting unnecessary notifications to other users (e.g., family members).

In some embodiments, sensor arrays 810 can include one or more sensors for measuring air quality. For example, sensor arrays 810 can include air pollution sensors and/or particulate sensors (e.g., laser sensors) for measuring an amount of particulate in the air within smart room 606. In some embodiments, sensor arrays 810 include volatile organic compound (VOC) sensors for measuring ambient concentrations of reducing gases (e.g., alcohols, aldehydes, ketones, organic acids, amines, organic chloramines, aliphatic, aromatic hydrocarbons, etc.) associated with poor air quality and/or smells. If sensor arrays 810 detect particulate levels or VOC levels above a threshold, smart room 606 may be configured to operate HVAC equipment to replace (i.e., exchange) the poor-quality air within smart room 606. More generally, smart room 606 may perform a “room air flush” to remove odors and improve air quality. In some cases, rather than controlling HVAC equipment directly, smart room 606 transmits a control signal to a BMS (e.g., BMS controller 366) to cause the BMS to operate the HVAC equipment. In some embodiments, a room air flush may cause HVAC equipment to exchange air at a maximum volume (e.g., based on equipment size) to replace the air in smart room 606 as quickly as possible. In some embodiments, fresh and/or incoming air may be scented while passing into smart room 606. In some embodiments, a room air flush may be manually activate, such as by facility staff (e.g., at a nurse's station) or via a smartphone app, as described below with respect to FIG. 9.

In some embodiments, smart room 606 includes a bedside terminal 812, which includes a user interface for receiving user commands and, in some cases, for displaying information. A user may interact with bedside terminal 812 to control various aspects of smart room 606, such as to turn on or off the lights, to raise or lower the temperature, etc. In a hospital, for example, bedside terminal 812 may also be used to call for a nurse, order a meal, make external phone calls, etc. In some embodiments, bedside terminal 812 is a computing device that includes at least a processor and memory for receiving and interpreting user inputs, and transmitting user input data to central computing system 602. For example, bedside terminal 812 may be a smart speaker that can detect audio (e.g., spoken words) from a user, and that can provide feedback via a speaker.

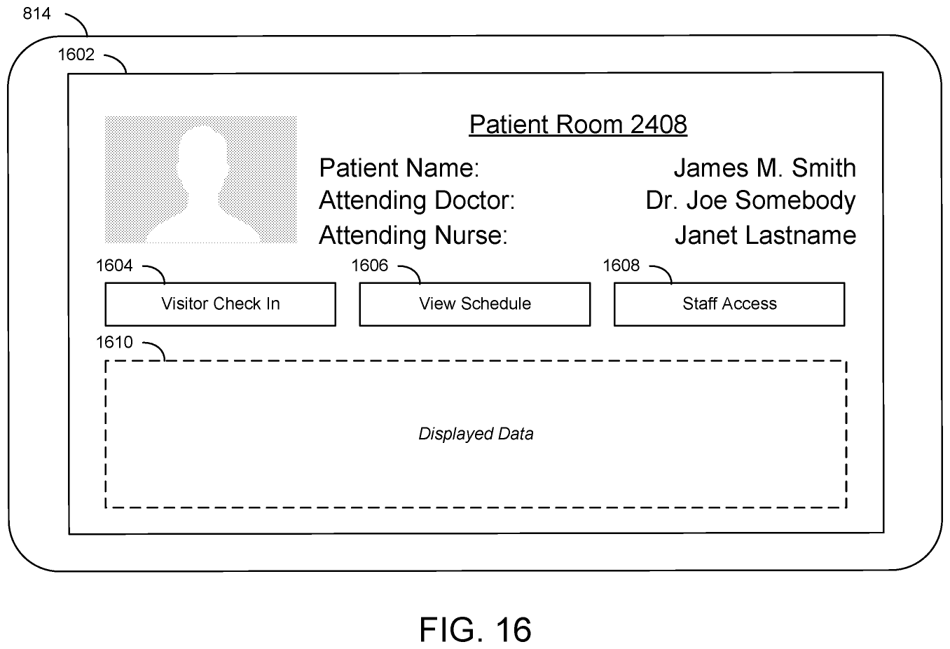

In some embodiments, smart room 606 also includes an information panel 814, which can be positioned outside of smart room 606 (e.g., near entryway 802). Information panel 814 may include at least a processor and memory for receiving and interpreting user inputs, and a user interface for presenting data. Information panel 814 may present any information relating to smart room 606, such that the information can be viewed by a user (e.g., a nurse or doctor) without entering smart room 606. For example, a user may view a schedule for the room, the current occupancy of the room, current room temperature, etc., via information panel 814. Additional features of information panel 814 are described in detail below, with respect to FIG. 16. In some embodiments, the information panel can include a display and/or interface inside the smart room 606, outside the smart room 606, or both inside and outside the smart room 606.

Smart room 606 may also include additional features that may be enabled by system 600 and the various components described above. In some embodiments, smart room 606 may include adjustable lighting that can be controlled by an occupant or that is adjusted automatically based on a detected mood or condition of the occupant(s). For example, smart room 606 may be configured to detect when a patient in a hospital is tired and may adjust the lighting accordingly, by closing blinds to limit ambient light and/or by adjusting the intensity or color of various lights within the room. In some embodiments, smart room 606 is also configured to play music or sounds based on an occupant's request, or based on a current mood/condition of the occupant. For example, a patient that is recovering from surgery may benefit from soothing sounds or music, and aromatherapy. Smart room 606 may be configured to detect the occupant's mood/condition and play appropriate sounds or music, initiate aromatherapy by controlling a scent diffuser, increase oxygen levels in the smart room, display soothing or encouraging messages via the display 808, change the color of lighting the smart room 606, adjust the bed 804, and/or otherwise manipulate the furniture and features of the smart room 606 to improve the occupant's experience.

In some embodiments, such as in a hospital, smart room 606 may also be configured to adjust meal or care timing for a patient based on a determination that the patient is resting, active, with visitors, etc. For example, if smart room 606 determines that the patient is interacting with visitors during a normal meal time, central computing system 602 may automatically adjust the patient's meal time based on the patient's schedule (e.g., to a time period when no visitors are scheduled). In some embodiments, smart room 606 may also be able to present movies, pictures, or music (e.g., via display 808) based on actions occurring within the room. For example, smart room 606 may be configured to present family pictures via display 808 to improve a patient's mood.

In some embodiments, smart room 606 is configured to detect when a patient returns from surgery, therapy, etc., and may welcome the patient back. For example, the patient's schedule may be analyzed to determine when the patient is returning from an appointment. In some embodiments, the patient's schedule may be analyzed in combination with occupancy detection to determine when the patient is back in smart room 606. In some embodiments, remote systems and devices 608 described above with respect to FIG. 6 can include external data systems that can access, or that maintain records relating to, an occupant's social media accounts, search history, purchasing preferences, etc. Accordingly, smart room 606 may be configured to determine an occupant's preferences to select appropriate music or movies, or to display images and messages via display 808.

In some embodiments, the actuators and elements of the room are controlled according to the user but temperature, pressure, and humidity are maintained within predetermined ranges. For example, the blinds may be opened according to user preferences, and the resulting affect on temperature, pressure, and humidity are monitored and the other systems of the room (e.g., the HVAC and air purity systems) are adjusted to maintain the temperature pressure and humidity within the predetermined range. In some embodiments, the BMS utilizes a machine learning engine or other artificial intelligence to learn the interrelationships between actions within a room or building to better adapt to user demands while maintaining temperature, pressure, and humidity within the predetermine range.

In some embodiments, the operation of the smart room systems 802-814 are controlled based on the SOM score determined by the PPMS 722 as discussed above. The SOM score can be an input of the smart room control application 912 discussed in detail below. In some embodiments, the SOM score can prompt an increase/decrease in temperature and the BMS 366 or another room control system will adjust HVAC settings to maintain a temperature, pressure and humidity (TPH) range/balance as required by a compliance standard. In some embodiments, the SOM score can prompt the opening of blinds or lighting levels in an attempt to improve the SOM score of the patient. In some embodiments, the SOM score can prompt a notification to the nurse call system or other scheduling system to initiate a nurse visit, a visit by an advisor, or another clinician. In some embodiments, the SOM score can prompt an identified individual (e.g., a friend or family member) to connect with the patient via a companion application hosted on a user device 604 or a remote system 608. For example, if the SOM score is dropping quickly, a prompt may be sent to all individuals identified by the patient and the prompt may inform the identified individuals that the patient needs encouragement or contact from a cared for person. This prompted connection can help improve the SOM score of the patient and therefore improve the patient's experience while staying in the smart room 606.

In some cases, TPH for a room and/or a building is monitored and checked for compliance with regulations or process controls. Regulations may include compliance standards set by governmental or non-governmental entities and compliance may be checked by a compliance officer. The compliance standards are generally set by outside organizations and define an acceptable range of environmental parameters. For example, hospital environments have defined temperature, pressure, and humidity (TPH) requirements defined in the compliance standards that must be maintained. Checks for compliance of TPH with the compliance standards may be checked randomly or on a set routine or schedule and can affect the ability of the building to continue operation (e.g., an out of compliance hospital may be inhibited from providing patient care).

In some embodiments, particularly for a building that serves as a hospital, The Joint Commission (TJC) may administer the compliance checks. In some such embodiments, if the hospital building or a room within the building (e.g., a patient room, an operating room, etc.) is found to be out of compliance (i.e., does not meet the compliance standards), a finding is identified and reported to the Centers for Medicare and Medicaid Services (CMS), who then perform an independent inspection of the building or room. Should the building or room fail the CMS inspection, a deemed status of the hospital may be lost. The loss of deemed status can result in the withholding of Medicare and/or Medicaid to the hospital. In a hospital setting, response to issues affecting TPH in a timely manner is critical.

The ability to control operation of the smart room systems 802-814 (i.e., building devices) based on the SOM score determined by the PPMS 722 while maintaining the rooms of the healthcare facility within the TPH or any other compliance standards allows for an improvement in patient satisfaction and SOM without sacrificing the health and compliance of the facility.

### Application for Enabling Smart Features

Referring now to FIG. 9, a block diagram of a user device 604 that includes a control application for a smart room (e.g., smart room 606) is shown, according to some embodiments. In some embodiments, user device 604 is a personal computing device belonging to an occupant of building 10. For example, user device 604 may be a smart phone or tablet belonging to a building employee or a visitor of the building. Users may interact with user device 604 to view and/or request information, input commands, and perform other functions associated with building 10. In particular, user device 604 may include an application that allows a user to control parameters or functions of smart room 606, or other components of system 600, to reduce user wait times (e.g., for an appointment or meeting), improve navigation to and within building 10, and otherwise automate one or more features to improve the user's experience.

User device 604 is shown to include a processing circuit 904, which includes a processor 904 and memory 910. It will be appreciated that these components can be implemented using a variety of different types and quantities of processors and memory. For example, processor 904 can be a general purpose processor, an application specific integrated circuit (ASIC), one or more field programmable gate arrays (FPGAs), a group of processing components, or other suitable electronic processing components. Processor 904 can be communicatively coupled to memory 910. While processing circuit 902 is shown as including one processor 904 and one memory 910, it should be understood that, as discussed herein, a processing circuit and/or memory may be implemented using multiple processors and/or memories in various embodiments. All such implementations are contemplated within the scope of the present disclosure.

Memory 910 can include one or more devices (e.g., memory units, memory devices, storage devices, etc.) for storing data and/or computer code for completing and/or facilitating the various processes described in the present disclosure. Memory 910 can include random access memory (RAM), read-only memory (ROM), hard drive storage, temporary storage, non-volatile memory, flash memory, optical memory, or any other suitable memory for storing software objects and/or computer instructions. Memory 910 can include database components, object code components, script components, or any other type of information structure for supporting the various activities and information structures described in the present disclosure. Memory 910 can be communicably connected to processor 904 via processing circuit 902 and can include computer code for executing (e.g., by processor 904) one or more processes described herein.

Memory 910 is shown to include a smart room control application 912, which may be a smart phone application, a software application, or other similar component that can be executed by processing circuit 904 to provide specific functionality. For example, application 912 may be executed in response to a user selecting an icon from a user interface of user device 604 (e.g., user interface 932, described below). In response to the user selecting the icon (e.g., from a home screen or an initial display on the user interface), application 912 may be executed (i.e., launched).

In some embodiments, a user may be required to log in after launching application 912. For example, after the user selects an icon for application 912, the user may be presented with a log in screen that prompts the user to input credentials, such as a user name, email address, and/or password. User inputs to the log in screen may be validated (e.g., by central computing system 602) to authenticate the user and allow the user access to application 912. In some embodiments, a user may also be able to generate a new profile or register to use application 912 via the log in screen.

Application 912 is shown to include a number of different functions that a user may access after the application is launched and/or the user logs in to application 912. Specifically, application 912 can including a schedule function 914 configured to manage (e.g., track and update) a schedule for a user of user device. In some embodiments, schedule function 914 may present the user with a schedule based on the user's records (e.g., user records 724), which may be stored locally or may be retrieved from central computing system 602. For example, user device 604 may communicate with central computing system 602 via network 446 to retrieve, in real time or at regular intervals, scheduling information (e.g., from schedule manager 714). The user's schedule may be presented as a graphical element via user interface 932.

In some embodiments, schedule function 914 may allow a user to modify the schedule by scheduling new events, deleting or cancelling scheduled events, changing event times, etc. For example, a patient of a hospital may access schedule function 914 to view and/or change a scheduled appointment time, or to cancel an upcoming appointment. In another example, a nurse working at a hospital may access schedule function 914 to view time periods where they are scheduled to work, to request time off, etc. In some cases, building staff (e.g., doctors, nurses) may also be able to view schedules for certain rooms of building 10 via schedule function 914, or may be able to view daily/weekly/monthly schedules that indicate schedules appointments with customers or patients.

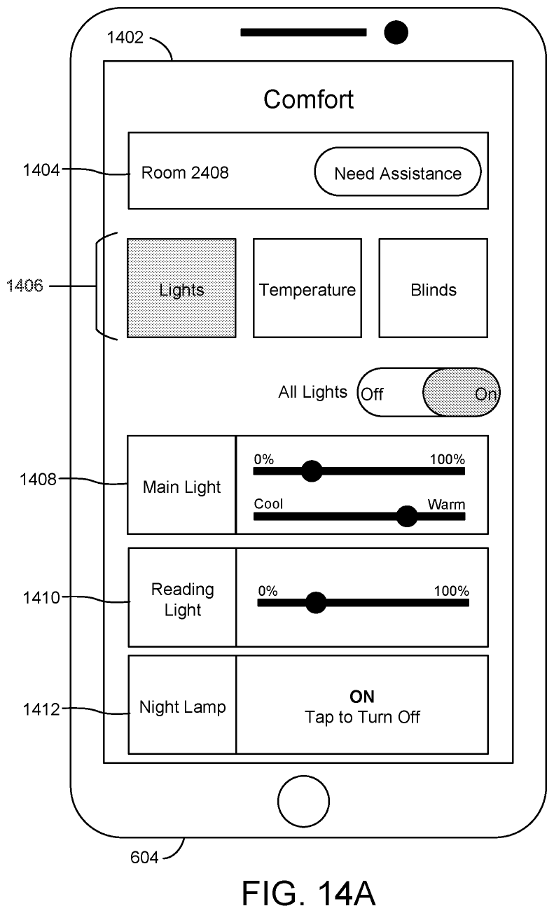

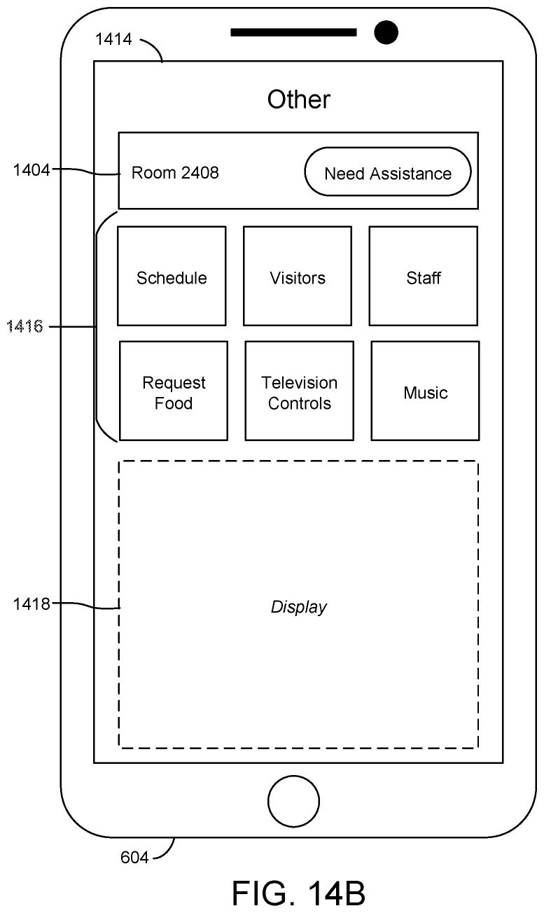

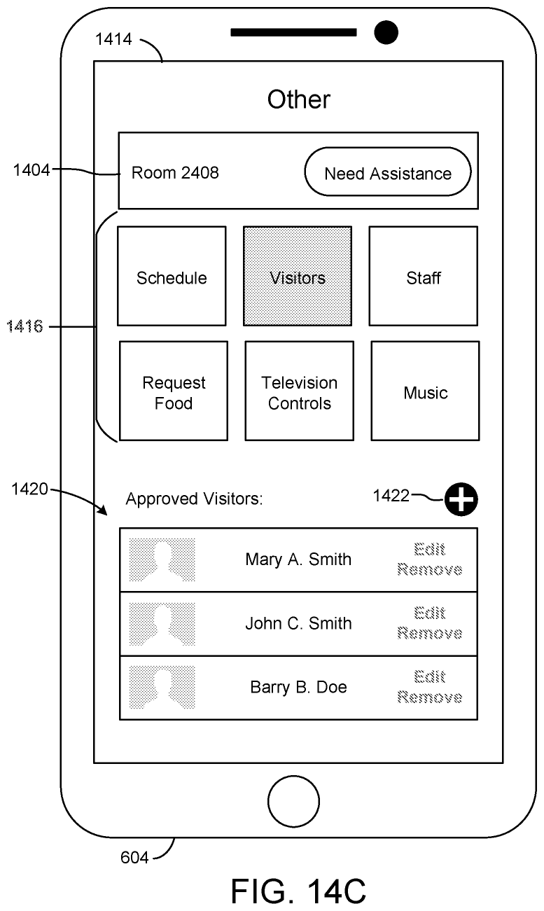

A room control function 916 may be selected to present (e.g., via user interface 932) a plurality of controls for a specific area or room within building 10. In some embodiments, room control function 916 is configured to determine the user device's current position within building 10 to detect the particular room the user wishes to control. In other embodiments, the user may manually enter a location (e.g., a room number) and room control function 916 may be dynamically configured to control parameters in the identified location. In any case, room control function 916 may allow the user to modify various parameters of a room (e.g., smart room 606), including but not limited to noise levels, lighting, climate, etc. For example, the user may access room control function 916 to change the temperature of the room, or to open or close the blinds in the room. Various other room control features are described in detail below, with respect to FIGS. 14A-14C.

A navigation function 918 may be configured to request navigation data (e.g., a map and/or route instructions) from a current location of user device 604 and/or from another predetermined location (e.g., a home address of a user) to a destination (e.g., building 10). In some embodiments, navigation function 918 transmits location information, such as a current location of user device 604, to navigation engine 716 of central computing system 602 to request navigation data. Navigation engine 716 may generate a route or other navigation data, which is then transmitted to user device 604. Navigation engine 716 may interpret the navigation data and may present the navigation data in a map, as text or audio-based directions, or in another format.

In some embodiments, navigation function 918 is also configured to generate and/or request navigation data for navigation within building 10. For example, navigation function 918 may provide a map or other route instructions that direct a user to particular areas (e.g., rooms) within building 10. In some embodiments, navigation function 918 may determine a current position of user device 604 within building 10, and may generate a route through the building to a destination or point of interest. For example, a patient in a hospital may be presented with navigation instructions to a particular examination room upon checking in for an appointment.

In some embodiments, navigation function 918 generates navigation data in response to a user input. For example, a user may manually request navigation and, in response, navigation function 918 may generate or request navigation data and present the navigation data to the user. In some embodiments, navigation function 918 may automatically generate navigation data based on a user's schedule, as determined by schedule function 914. In such embodiments, navigation function 918 may automatically generate or request navigation data from a current location of user device 604 or from a user-defined location to building 10 and/or to a particular room in building 10 based on a determination that the user has an upcoming appointment. For example, this automatic navigation data generation may occur a predetermined amount of time prior to the user's appointment, to allow the user enough time to navigate (e.g., drive, walk, etc.) to the destination. In this regarding, navigation function 918 may also be configured to determine the user's current location in relation to the destination, and can display navigation information based on when the user would need to leave to make a schedule appointment.

A parking/valet function 920 may allow a user to reserve parking or request valet service at the destination (e.g., building 10). In some embodiments, parking/valet function 920 may automatically prompt the user to reserve parking or request a valet in response to determining that the user has an upcoming appointment, or in response to the user requesting navigation data. In some embodiments, the user can manually request parking or valet service. Parking/valet function 920 may be configured to determine a destination for the user, identify a closest parking structure to the destination, and reserve parking within the identified parking structure. In some embodiments, parking/valet function 920 can also reserve a particular spot within the parking structure. Thus, in some embodiments, navigation function 918 may be configured to generate a route directly to the particular spot.

In some embodiments, parking/valet function 920 may prompt a user for payment in response to a parking/valet request. For example, parking/valet function 920 may display (e.g., via user interface 932) a payment window that allows the user to input payment information to reserve parking or pay for valet service. In some such embodiments, parking/valet function 920 may communicate either direct with, or through central computing system 602, a remote or third-party payment system (e.g., one of remote systems and devices 608) to facilitate payment of a parking or valet fee. Once the user has reserved parking/valet service and/or has made a payment, parking/valet function 920 may be configured to generate a token or code, or can request and token/code from central computing system 602, that allows the user to access the parking structure.

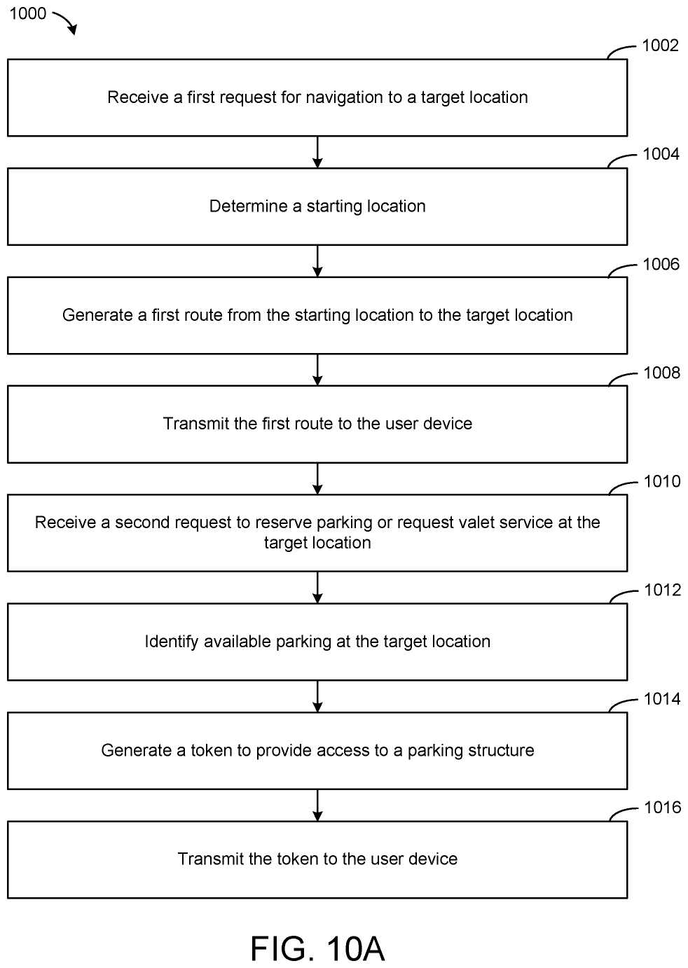

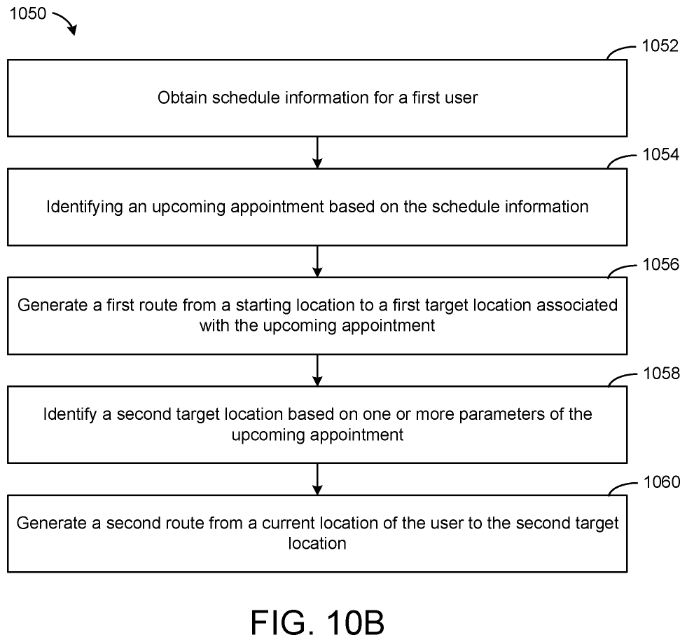

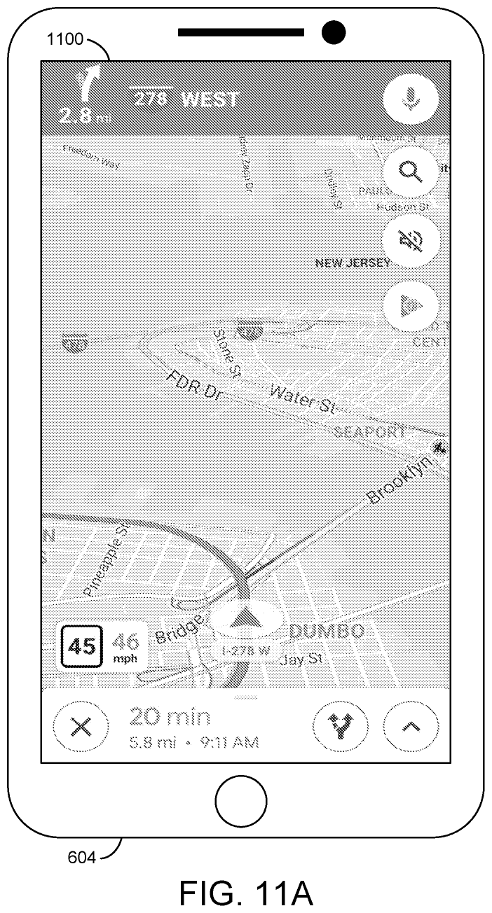

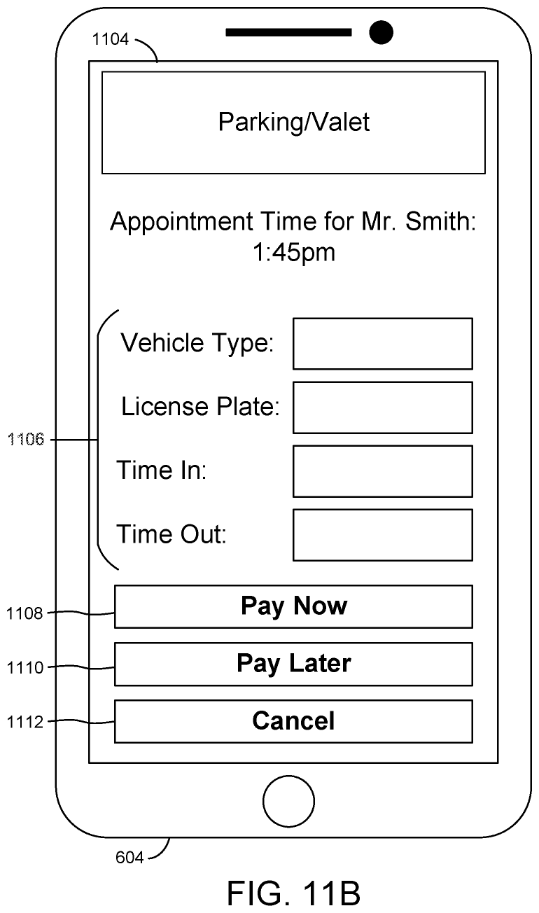

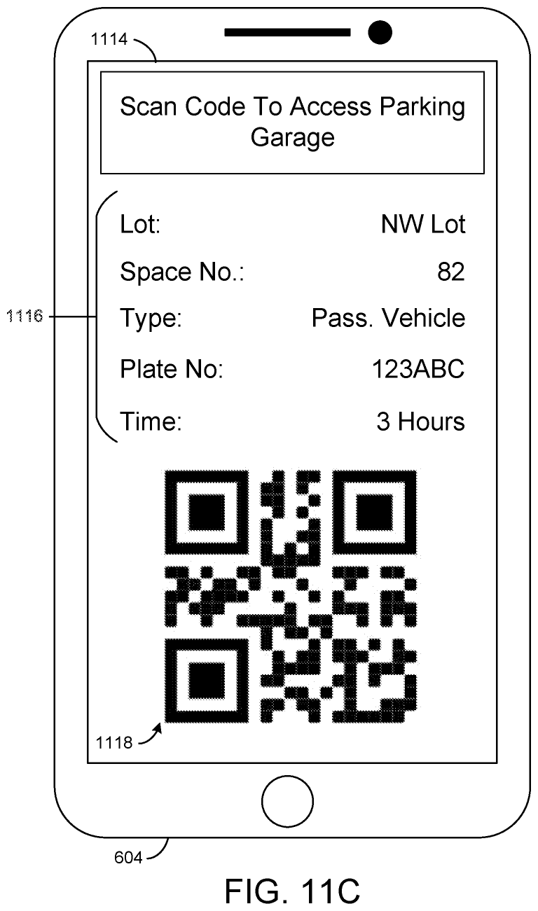

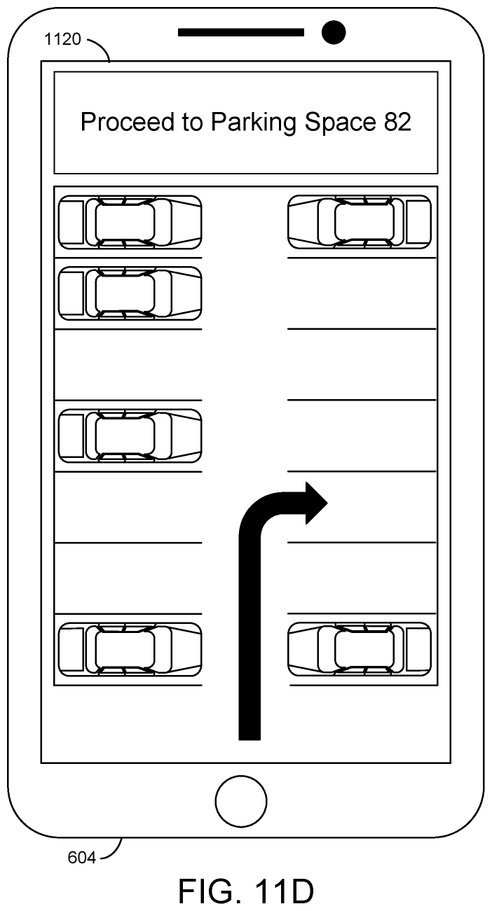

In some embodiments, parking/valet function 920 communicates with other remote or third-party systems, such as a management system for a pharmacy (e.g., in a healthcare facility). In particular, a patient's prescription information may be automatically transmitted to the pharmacy's system (e.g., during or after the patient's appointment). Subsequently, when the patient checks-out from an appointment and/or requests their vehicle from a valet service, parking/valet function 920 may transmit an indication at a prescription is required to valet staff and/or a parking lot attendant. Accordingly, the valet staff and/or a parking lot attendant may retrieve the patient's prescription, which can then be handed to the patient when they retrieve their vehicle and/or exit a parking structure. Additional features of parking/valet function 920 are described in greater detail below, with respect to FIGS. 10-11D.

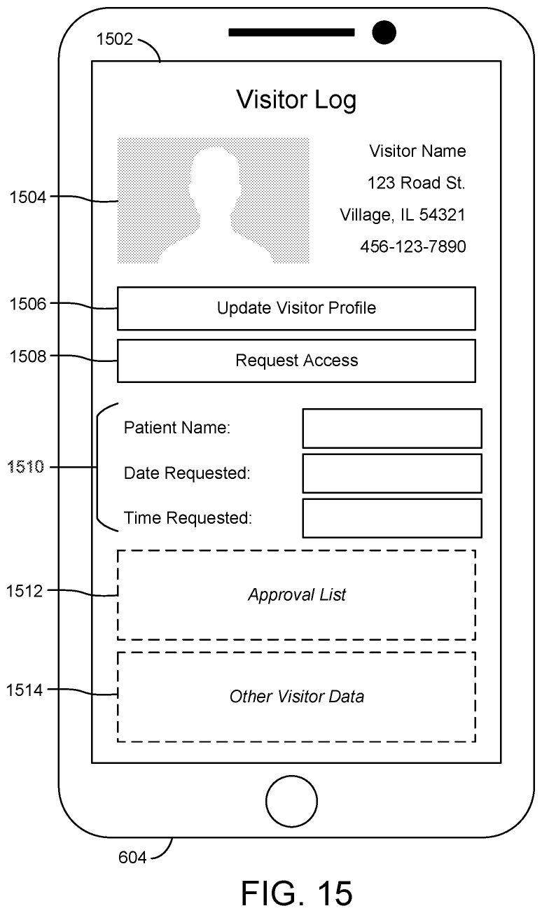

Application 912 is also shown to include a visitor control function 922 configured to track and/or manage visitors to a room occupied by a user of user device 604. In particular, the user may be able to approve or reject visitor requests, and may be able to check-in visitors via application 912. In some embodiments, visitor control function 922 allows the user to define a list of approved visitors that can be pre-registered to save time when visiting the user of user device 604 within building 10. In a hospital, for example, a patient may register a number of visitors (e.g., family members) for visitation rights, such that the registered visitors may be automatically checked-in upon arrive to the hospital and/or granted access to a room (e.g., smart room 606) that the patient is assigned to. Various other visitor control functions are described in detail below with respect to FIG. 15.

In some embodiments, application 912 may include access control functions that enable user device 604 to act as a keycard or security badge, allowing accessed to locked rooms or certain areas of a building. In this example, a user may be able to swipe or tap user device 604 against an access control device (e.g., an RFID receiver) to transmit security information (e.g., a token), allowing access to an area or unlocking a door. It will also be appreciated that application 912 is not limited to only the features described above, but that application 912 may include other features not shown in FIG. 9.

Still referring to FIG. 9, memory 910 is shown to optionally include a family engagement application 924. Specifically, family engagement application 924 may be optionally installed on user device 604 based on an implementation or use of user device 604. For example, family engagement application 924 may not be installed on a user device operated by a patient in a hospital, but may be installed on a user device operated by a family member of the patient. Likewise, family engagement application 924 may not be installed on user device 604 when user device 604 is not utilized in a hospital setting. Family engagement application 924 may provide a variety of functionality to visitors and/or family members of a patient (e.g., staying in smart room 606). For example, family engagement application 924 may receive and display notifications when a physician, nurse, or other hospital staff member enters a patient's room, as described above.

In some embodiments, family engagement application 924 provides an interface for family members and/or visitors to ask questions to an attending physician or care team. Accordingly, family engagement application 924 may include a chat or messaging interface to facilitate secure communications between a user device operated by the family members or visitors and a member of the care team. In some embodiments, family engagement application 924 can interface with central computing system 602 to determine a various information about a patient, such as the patient's schedule, a most recent care team member that visited the patient, etc. This patient-related information can then be presented via an interface of a user device operated by the family members or visitors, providing additional insight into the treatment and care of the patient.

In some embodiments, family members may submit and/or record observations regarding the patient via family engagement application 924, which may be particularly useful in pediatrics. For example, a family member may record changes in mood, appetite, temperature, etc., associated with the patient, to provide greater insight to the care team in treating the patient. In some embodiments, family engagement application 924 is configured to provide a map and/or directions of an interior of the facility (e.g., a hospital) to aid family members and/or visitors in finding particular spaces (e.g., the patient's room, a cafeteria, a coffee shop, etc.) with the facility. In this regard, family engagement application 924 may interface with navigation function 918 to provide intra-facility navigation.

In some embodiments, family engagement application 924 can include a digital whiteboard that provides a wide variety of patient information, similar to a whiteboard or other display maintained by hospital staff in the patient's room. In some such embodiments, the digital whiteboard may indicate names and contact information for care team members (e.g., physicians, nurses, etc.). In some embodiments, care team information may be automatically and/or regularly updated based on care team shifts or operating hours. In some embodiments, the digital whiteboard may indicate a care team member that was most recently in the patient's room (e.g., based on RTLS data, as described above with respect to FIG. 8). In some embodiments, the digital whiteboard also indicates the patient's schedule, care plan and goals, and pain level and/or status, along with a most recent physician visit time and additional contact information (e.g., for hospital staff) that the family of the patient can contact to ask questions.

Memory 910 is also shown to include a user interface (UI) generator 926 configured to dynamically generate, modify, and/or update graphical user interfaces that present a variety of data, including the various user interfaces described herein. For example, UI generator 926 may be configured to generate any of the user interfaces required for application 912, including a schedule interface, a navigation and/or parking interface, a visitor control interface, a room control interface, etc. UI generator 926 may be configured to generate graphical control elements that enable the various functions described above.

Memory 910 is also shown to include a language processing engine 928. Language processing engine 928 may be configured to process. Like language processing engine 720 described above, language processing engine 928 may include natural language processing (NLP), or may communicate with a remote and/or third-party NLP service, to process spoken commands received from occupants of a building. In some embodiments, audio data is received from a speaker or other audio device included in with user device 604 (not shown). The audio device (e.g., microphone) may be configured to continuously and/or occasionally monitor and/or record sounds (e.g., human speech), which is processed via language processing engine 928. Language processing engine 928 may include a neural network or artificial intelligence that can convert the audio data into text-based data or other similar data that can be interpreted by user device 604. For example, language processing engine 928 can convert the audio data into commands that enable features of application 912 and/or that control application 912.

It will be appreciated that, in some embodiments, language processing may be implemented exclusively by central computing system 602 and/or may be implemented by a remote and/or third-party service. In such embodiments, language processing engine 928 may simply preprocess the audio data and may transmit the audio data along with a processing request to central computing system 602 and/or the remote and/or third-party service. Language processing engine 928 may then receive converted data (e.g., commands) in return.

User device 604 may also include additional components, such as navigation components 930 and a user interface 932. Navigation components 930 may include one or more components or devices configured to determine a location of user device 604. In some embodiments, navigation components 930 include at least a GPS transceiver for determining a geographical location (e.g., latitude/longitude) of user device 604. In some embodiments, navigation components 930 may include a cellular transceiver or other radio transceiver for determining a location of user device 604 by triangulation of signals. In some embodiments, navigation components 930 may include a WiFi or Bluetooth® transceiver for detecting a location of user device 604 based on a distance from one or more WiFi or Bluetooth® transmitters. It will be appreciated that any other suitable component(s) for determining a location of user device 604 are contemplate herein (e.g. LIDAR).

User interface 932 may be any component that allows a user to interact with user device 604. In some embodiments, user interface 932 includes a screen for displaying information and/or graphics. In some such embodiments, user interface 932 may be a touchscreen capable of receiving user inputs. In some embodiments, user interface 932 includes a user input device such as a keypad, a keyboard, a mouse, a stylus, etc.

Still referring to FIG. 9, user device 604 is also shown to include a communications interface 940. Communications interface 940 may facilitate communications between user device 604 and any of the other components of system 600. For example, communications interface 940 may allow user device 604 to transmit data to central computing system 602 and to receive data from central computing system 602 (e.g., via network 446). Accordingly, communications interface 940 can be or can include a wired or wireless communications interface (e.g., jacks, antennas, transmitters, receivers, transceivers, wire terminals, etc.) for conducting data communications.

In some embodiments, communications via communications interface 940 may be direct (e.g., local wired or wireless communications) or via network 446 (e.g., a WAN, the Internet, a cellular network, etc.). For example, communications interface 940 can include an Ethernet card and port for sending and receiving data via an Ethernet-based communications link or network. In another example, communications interface 940 can include a WiFi transceiver for communicating via a wireless communications network. In another example, communications interface 940 may include cellular or mobile phone communication transceivers. In some embodiments, communications interface 940 may be utilized in combination with navigation components 930 to determine a location of user device 604.

### Navigation and Parking

Referring now to FIG. 10A, a flow diagram of a process 1000 for providing navigation and parking data to a user device (e.g., the user device 604) is shown, according to some embodiments. Process 1000 may be implemented by system 600, in some cases, and more particularly may be implemented by central computing system 602 in combination with user device 604. Process 1000 may provide a user with relevant navigation and parking/valet information based on user requests (e.g., from application 912) and/or based on identified upcoming appointments associated with the user. In this regard, process 1000 may advantageously require very few user inputs to obtain and present this information, which may provide users (e.g., customers, patients) with an intuitive and all-in-one solution for navigating to a target location (e.g., building 10) for an appointment. It will be appreciated that certain steps of process 1000 may be optional and, in some embodiments, process 1000 may be implemented using less than all of the steps.

At step 1002, a first request for navigation is received. In some embodiments, the first request is manually entered by a user, such as via user interface 932 of user device 604. In such embodiments, the user may enter a current or starting location and a target location (e.g., site 500), and may subsequently request navigation to the target location. Thus, the first request may be transmitted from user device 604 to central computing system 602. In some embodiments, the starting location may be a previously entered and/or user defined location, such as a home address. In other embodiments, the starting location may be automatically determined based on a location of the user's device (e.g., user device 604) at step 1004.

In some embodiments, the first request is received in response to a determination that the user (e.g., of user device 604) has an upcoming appointment. For example, a schedule manager (e.g., schedule manager 714) for a hospital may determine that a first patient has an upcoming appointment, and may automatically request (e.g., from navigation engine 716) navigation data from the user's current location to the hospital. In some such embodiments, central computing system 602 may query a current location from user device 604 prior to generating navigation data, and in response to receiving the first request. In some embodiments, a notification is provided to the user device 604 at predetermined amount of time (e.g., fifteen minutes) before a recommended departure time accounting for appointment time, travel time, check in time, etc. such that the user has adequate time to arrive in a timely manner for the appointment. Integration with the hospitals scheduling system provides advantageous efficiency.

At step 1004, a current location of the user device is determined. As mentioned above, the current location of the user device may be determined based on a user-initiated navigation request or based on an automatically generated navigation request. In any case, a central computing system or server (e.g., central computing system 602) may query the user device (e.g., user device 604) for current location data, such as by transmitting a prompt to the user device. The user device may utilize navigation components (e.g., navigation components 930) to determine its current location, which is then transmitted to the central computing system. In some embodiments, the current location data includes a latitude and longitude, a street address, or other indication of a current geographical position of the user device. For example, the current location data can include GPS data providing coordinates for the user device.

At step 1006, a first route from the starting location to the target location is generated. In some embodiments, the first route includes a set of turn-by-turn instructions that can guide a user of the user device to the target location. For example, the first route may include instructions such as “Turn left in 800 feet,” or “Take a right on Main Street.” In some embodiments, the first route includes a map that graphically indicates the route to the target location. In this regard, the user may follow the first route using the map. In some embodiments, both a map and turn-by-turn instructions are generated. In some embodiments, the first route may be generated based on user-defined constraints. For example, a user of the user device may indicate that the generated route should be the fastest route to the target location, or the shortest route in the target location. In another example, the user may indicate that generated routes should avoid toll roads.

At step 1008, the first route is transmitted to the user device. In some embodiments, the first route is generated by a central computing system or server, associated with the target location and/or third-party/remote, and transmitted via a wireless network (e.g., network 446) to the user device. The user device may receive the first route (i.e., navigation) data and may display the first route on a user interface. For example, the user device may display the map and turn-by-turn directions. In some embodiments, the user may be able to modify the first route via the user interface, such as by avoiding particular streets, adding stops, etc. The generation and display of a first route is described in greater detail below with respect to FIG. 11A.

At step 1010, a second request to reserve parking and/or to request valet service is received. In some embodiments, the second request is received in response to the user requesting navigation (e.g., the first request). For example, the user may request navigation to the target location, and either prior or subsequently to generating the navigation data (e.g., the first route), the user may be prompted to reserve parking at the target location. Accordingly, it will be appreciated that in some embodiments, steps 1010-1016 of process 1000 may be executed prior to, concurrently with, or subsequent to steps 1002-1008. In some embodiments, the second request is received from a user device, such as by the user selecting a graphical element of a user interface. In some embodiments, rather than receiving a second request, a central computing system may automatically generate and transmit a notification to the user device, prompting the user to purchase/reserve parking or valet service.

At step 1012, available parking is identified at the target location. In some embodiments, identifying available parking includes identifying a parking structure (e.g., a parking garage or lot) near the target location. For example, a site (e.g., site 500) may include multiple parking structures, and the parking structure nearest the target location (e.g., a particular portion or entrance to a building) may be identified. In such embodiments, available parking may be identified based on one or more constraints, such as a distance to the target location. In some embodiments, a particular parking space within a parking structure may also be identified. In some embodiments, the parking structure is assigned to the user and access is provided to the assigned parking structure. In some embodiments, the identified parking structure is provided as a recommended parking structure and the user is provided access to all parking structures.

At step 1014, a token is generated to provide access to the identified parking structure. The token may include a digital code (e.g., a string of alphanumeric characters), a bar code, a QR code, or any other similar type of code or token. In some embodiments, the token is generated by a central computing system in response to identifying available parking. In other embodiments, a notification is first generated and transmitted to the user device, prompting the user to provide payment information to pay for the parking or valet service. In such embodiments, the token may be generated only after the user submits payment information and/or the payment is verified. In some embodiments, subsequent to the user providing payment to reserve parking or valet service, the first route may be modified to replace the originally target location with a new target location based on the identified or reserved parking structure or particular parking space. In this manner, the user may be directed to the reserved parking space or to a valet station rather than to the original target location.

At step 1016, the token is transmitted to the user device. In some embodiments, the token is generated by a central computing system or server and transmitted via a wireless network (e.g., network 446) to the user device. The user device may receive the token and may store the token for future use (e.g., when arriving at the target location). In some embodiments, upon arriving at the parking structure, the user may access the user device to display the token or transmit the token to a parking structure access control device (e.g., access control point 518). In some such embodiments, the user may present the displayed token (e.g., a bar code) to a scanner associated with the access control point, or may wireless transmit the token to the access control point via a Bluetooth®, WiFi, RFID, or other similar connection. Accessing the parking structure using the token is described in greater detail below with respect to FIG. 11C.

Referring now to FIG. 10B, a flow diagram of a process 1050 for providing navigation data to a user device (e.g., the user device 604) based on an upcoming appointment is shown, according to some embodiments. Like process 1000, process 1050 may be implemented by system 600, in some cases, and more particularly may be implemented by central computing system 602 in combination with user device 604. Advantageously, process 1050 may automatically generate navigation data based on a user's schedule, requiring little to no user input.

Process 1050 may also dynamically update navigation data when secondary target locations are identified. For example, a user traveling to a hospital for a flu vaccine appointment may be automatically rerouted to another, nearby hospital or other facility that has a shorter wait time, thereby reducing the total appointment time for the user. Process 1050 may therefore allow facilities to utilize capacity more efficiently. In a hospital, for example, clinicians with downtime due to empty time periods on their schedules can pick up additional patients that would otherwise have to wait for or cancel an appointment at a first location, leading to increased revenue and improved patient care. It will be appreciated that certain steps of process 1050 may be optional and, in some embodiments, process 1050 may be implemented using less than all of the steps.

At step 1052, a user's schedule information is obtained. In some embodiments, the user's schedule information is obtained (e.g., retrieved or received) by schedule function 914, such as from schedule manager 714. In other embodiments, the user's schedule information is obtained from a local (e.g., managed by the user on user device 604) or online calendar. In any case, the user's schedule information may indicate one or more upcoming appointments or events that were either entered by the user, received from an external system (e.g., a hospital's scheduling system), or both. For example, the user's schedule information may indicate multiple blocks of time that the user is busy with appointments, events, etc.

At step 1054, an upcoming appointment is identified based on the user's schedule information. The upcoming appointment may include relevant appointment information, such as a start and end date/time, an indication of the type of appointment (e.g., a meeting, a doctor or dentist appointment, etc.), a location of the appointment, etc. Accordingly, in some embodiments, the upcoming appointment is associated with a particular location (e.g., a first target location), such as a building or street address. For example, an upcoming doctor's appointment may include a location of the hospital and/or an indication of a room number where the appointment will be conducted. In some such embodiments, the location associated with the upcoming appointment may be automatically determined, such as based on information received from an external scheduling service (e.g., a hospital's scheduling system), or the location may be entered by the user when creating the appointment (e.g., in a calendar).

In some embodiments, the upcoming appointment is automatically identified a predetermined amount of time prior to the start of the appointment. For example, the appointment may be identified at least 30 minutes prior to the start of the appointment. The predetermined amount of time may be defined and/or modified by the user (e.g., during the creation of an appointment), or the predetermined amount of time may be dynamically adjusted based on a travel time to the appointment. In particular, the upcoming appointment may be identified early enough that the user can navigate (e.g., drive, walk, take public transport, etc.) to the appointment on time. For example, it may be determined that the travel time to the upcoming appointment is 15 minutes based on current traffic, a starting location, etc., and thus the upcoming appointment may be identified at least 15 minutes before the scheduled start time. Accordingly, in some embodiments, step 1054 may be performed concurrently with step 1056, as described below. In some embodiments, the user enters a desired service at step 1054 (e.g., a urgent care visit, a vaccination appointment, etc.) that did not previously exist in the user's schedule. In some embodiments, the desired service can be selected within an application or via an interface such that the schedule manager 714 is automatically updated with the desired service.

At step 1056, a first route from a starting location to a first target location is generated. As described briefly above, the first target location may be the location associated with the upcoming appointment, such as a particular building or address. Similar to step 1006 of process 1000, the first route can include a set of turn-by-turn instructions that can guide a user of the user device to the first target location. For example, the first route may include instructions such as “Turn right in 0.5 miles.” In some embodiments, the first route includes a map that graphically indicates the route to the first target location. In this regard, the user may follow the first route using the map. In some embodiments, both a map and turn-by-turn instructions are generated. In some embodiments, the first route may be generated based on user-defined constraints. For example, a user of the user device may indicate that the generated route should be the fastest route to the first target location, or the shortest route in the first target location. In another example, the user may indicate that generated routes should avoid toll roads.

At step 1058, a second target location is identified based on one or more parameters of the upcoming appointment. The second target location may be a building, address, etc., that is different from the first target location, and the one or more parameters may include a type of appointment or service, an appointment time, etc. For example, the first and second target locations may be a different hospital that provides at least one common service (e.g., pediatrics, radiology, etc.). In particular, the second target location may be determined to offer a similar appointment type or service as the first target location, but with a shorter wait. For example, it may be determined that the user would need to wait over 30 minutes for their appointment at the first target location (e.g., due to other appointments that ran long, due to staffing issues, etc.), and thus the second target location may be identified as providing the same appointment type or service with a shorter wait.

In addition to scheduling information, in some embodiments, building data from the first and/or second target locations is utilized to identify the second target location. For example, the second target location may be identified based on an indication from occupancy sensors, security cameras, etc., of the first target location, which indicate that certain areas of the building (e.g., waiting rooms) are at or near capacity, or that these areas have over a threshold number of occupants. In this example, the number of occupants in a waiting room may provide an indication of an expected wait time for new patients. Similarly, building data (e.g., occupancy sensor data, video feeds, etc.) from the second target location may indicate that waiting rooms or other areas of the second target location are not full or that these areas have a suitable amount of capacity. Thus, in this example, the second target location may be determined based on a wait time predicted by the current occupancy of the second target location.

In some embodiments, the second target location is identified as the user is navigating (e.g., driving, riding, walking, etc.) to the first target location. For example, the user may be driving to the first target location (e.g., a hospital) for a vaccine appointment when the second target location is identified. In this example, it may be determined that the first target location had a significant wait time or that the first target location had run out of available vaccines, although it will be appreciated that any of a number of other situations may arise that would require rescheduling of an appointment. Accordingly, in some embodiments, the second target location is identified from one or more suitable locations (e.g., other hospitals) within a predefined distance of the first target location or the current location of the user (e.g., based on location data from user device 604). For example, only locations within a predefined number of miles or a predefined drive time may be eligible for identification as the second target location. In some embodiments, the predefined distance may be determined in part by a current wait time at the first target location. For example, a first time to service is calculated by summing the current travel time to the first target location and a projected wait time at the first target location, and a second time to service is calculated by summing a travel time to the second target location and a projected wait time at the second target location. If the second time to service is less than the first time to service, the system provides a recommendation to the user to reroute to the second target location.

In some embodiments, the user (e.g., of user device 604) can also be presented with a prompt to accept or decline the change in target location. In some embodiments, the prompt is displayed via a screen of the user's device (e.g., user interface 932 of user device 604). Accordingly, the user may decide to decline the change in target location, thereby maintaining their appointment at the first target location. As an example, a user may choose to decline the change in target location because a preferred doctor is at the first target location, or because they simply prefer the first target location.

At step 1060, a second route to the second target location is generated based on a current location of the user. Accordingly, in some embodiments, step 1060 may be the same as or similar to step 1056. In particular, the second route can include a set of turn-by-turn instructions and/or a map for navigating to the second target location. In some embodiments, step 1060 may also include determining the current location of the user (e.g., based on location data from user device 604) prior to generating the second route. In this manner, the first route (e.g., directions and/or a map) may be dynamically updated and/or replaced to provide navigation to the second target location. Additionally, if an appointment is required at the second target location (e.g., a check in, a scheduled appointment slot, etc.) the system will automatically cancel the appointment at the first target location and schedule a new appointment at the second target location.

In some embodiments, such as in a hospital setting, a care team and/or hospital staff may be notified once a patient has arrived at the facility (e.g., after the token is transmitted to the user device at step 1016 of process 1000 or after the second route is generated at step 1060 of process 1050). For example, the patient's location may be determined based on GPS data from the patient's device (e.g., user device 604) and, once the patient is within a predefined distance of the target location (e.g., the hospital), the patient's device may be configured to generate and transmit a notification (e.g., a text message, a push notification, a voice call, etc.). In another example, the care team and/or hospital staff is notified once the patient provides a token to an access control device at a parking structure.

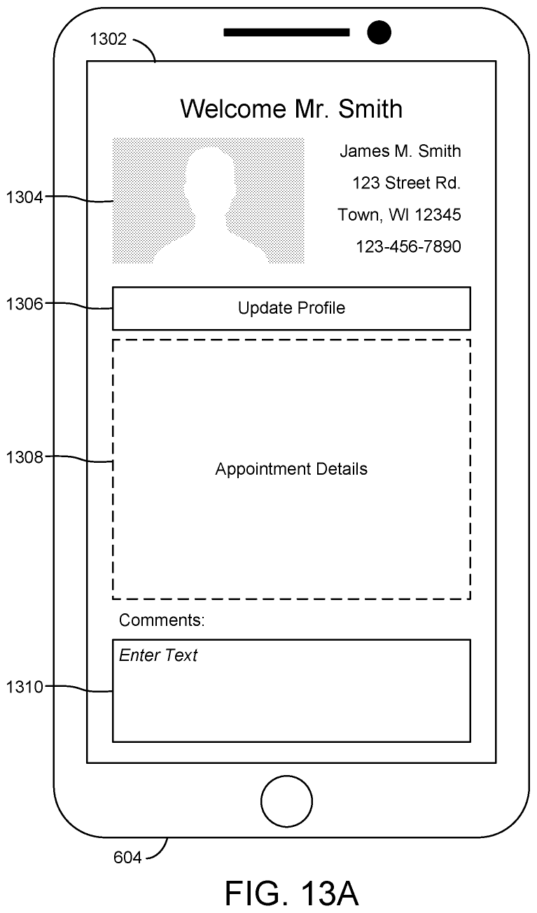

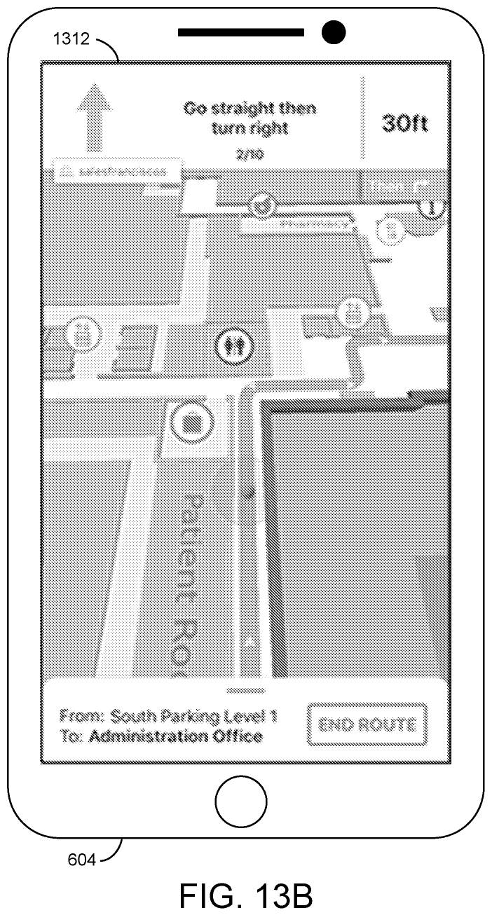

Referring now to FIGS. 11A-11D, example interfaces for presenting navigation and parking data are shown, according to some embodiments. These example interfaces may be generated and/or displayed by a user device (e.g., user device 604) that includes a smart room control application or other similar application associated with smart features of a building (e.g., building 10). As shown, these interfaces may be displayed on a user interface of the user device, such that a user may interact with the interfaces by entering information, scrolling, resizing, or otherwise manipulating various graphical elements (e.g., icons, buttons, etc.). It will be appreciated that the interfaces shown in FIGS. 11A-11D, and the various other interfaces described herein with respect to FIGS. 13A-16, are examples of interfaces that can be generated and presented by the user device, but the particular design or layout of these interfaces is not intended to be limiting.

Turning first to FIG. 11A, a navigation interface 1100 is shown, according to some embodiments. As described above, interface 1100 may be displayed in response to a request for navigation based on a user input or based on a determination that the user has an upcoming appointment. Interface 1100 is shown to include a map that includes a highlighted route for the user to follow to reach a target location (e.g., building 10). In some embodiments, the user device (e.g., user device 604) may continuously update and/or determine its location, which can be utilized to update interface 1100. For example, interface 1100 may move or pan while the user drives to the target location.

In some embodiments, interface 1100 displays turn-by-turn directions, or at least an upcoming turn. As shown, for example, the user is instructed to take a slight right in 2.8 miles. Interface 1100 may also provide other information such as a speed limit and the user's current speed, road closures, accidents, etc. In some embodiments, the user may modify the route from interface 1100, such as by selecting an alternate route, changing the starting or target location, etc. In some embodiments, the user device may interface (e.g., communicate) with a third-party service for generating interface 1100. For example, interface 1100 may be generated by a navigation service such as Google Maps®. In other embodiments, interface 1100 is generated by central computing system 602 and/or user device 604.

In some embodiments, the navigation features described herein may be integrated into a multimedia system or a control system for a vehicle. In some such embodiments, central computing system 602 may generate and transmit user interfaces and/or navigation data to the multimedia system, for display on a user interface within the vehicle. In some embodiments, an autonomous or semi-autonomous vehicle may receive navigation data from central computing system 602, and may automatically navigate to the target location.

FIG. 11B shows a parking/valet interface 1104, according to some embodiments. Interface 1104 may be displayed in response to a user-input or based on a determination that the user has an upcoming appointment. Interface 1104 may prompt the user to input various information in order to reserve parking or request valet service. In the example shown, interface 1104 displays an upcoming appointment time for a user, and may also display other information such as an estimate time of arrival, a time that the user should leave by to make the appointment, etc. The user may then enter information into fields 1106, such as a vehicle type (e.g., “car,” “motorcycle,” etc.), a license plate number, an estimate time of arrival to the parking structure/valet station, and an estimated time of departure. In some embodiments, the user may enter a number of hours rather than an estimate departure time. In some embodiments, a time of arrive or “time in” may be automatically populated based on the estimated amount of time for the user to reach the parking structure or based on the appointment time.

After entering relevant information, which may include additional data other than fields 1106 shown in FIG. 11B, the user may select a “Pay Now” icon 1108 to pay for the reserved parking, if necessary. Selecting icon 1108 may navigate the user to a secondary interface that allows the user to input payment information. In some embodiments, selecting icon 1108 may navigate the user to a remote or third-party payment system. Once the user has paid for parking, the reservation may be confirmed. In some embodiments, the user may alternatively select a “Pay Later” icon 1110 that allows the user to reserve parking or valet service and pay at a later time, such as when entering or exiting the parking structure. The user may also choose to cancel the parking/valet request by selecting “Cancel” icon 1112.

In some embodiments, a process for paying for parking/valet service may be incorporated into a user (e.g., patient) check-out process. For example, a patient at a hospital may pay for parking while checking-out from an appointment or after an extended stay. In some such embodiments, the payment for parking/valet service is incorporated into a payment for other services, such as a co-pay for medical treatments and/or a payment for prescriptions. In this manner, a patient may be required to submit only a single payment that includes co-pays, prescriptions, and parking/valet, rather than conducting two or three separate transactions, thereby saving time and hassle for the patient. In some embodiments, a user may choose to defer payment for parking/valet service in such a manner (e.g., to perform a single transaction at check-out) by selecting icon 1110.

After paying for parking/valet service, if required, a parking interface 1114 may be displayed, as show in FIG. 11C. Interface 1114 may present reservation details 1116, such as a location or identifier for the assigned parking structure or valet station, a particular space number (if applicable), a confirmation of the vehicle type and plate number, and an indication of the reserved amount of time. It will be appreciated, however, that any additional information may also be presented. Additionally, interface 1114 includes a code 1118, such as a QR code, that can be displayed via a user interface of user device 604. In some embodiments, interface 1114 is accessed or displayed once the user reaches an access point to the parking structure or a valet station. The user can then scan code 1118 at the access point, or a valet can scan code 1118, to admit the user to the parking structure or to accept the user's vehicle for valet service. As discussed above, in some embodiments, interface 1114 does not include code 1118. Rather, an access code for the parking structure/valet is stored on user device 604 as a digital token that can be wirelessly transmitted to the valet or the access point.

In some embodiments, code 1118 and/or interface 1114 are generated as part of a user pre-registration process. For example, a patient may pre-register for an appointment at a hospital or a visitor to the hospital may pre-register for access to a particular patient's room. Pre-registration may include providing personal identifying information (PII), such as a user's name, address, phone number, billing information, etc. In some embodiments, a user may also provide various preferences during pre-registration, such as room temperature and humidity preferences as described above with respect to PPMS 722. In some embodiments, a user may also connect third-party accounts, such as social media accounts or bank accounts, to a facility's central computing system (e.g., central computing system 602). For example, a user may pre-register by connecting a Google® account to a building's computing system such that various information about the user (e.g., name, phone number, etc.) is automatically transferred to the building's computing system.

After pre-registering, code 1118 and/or interface 1114 may be displayed on the user's device and may act as a “fast pass” for checking the user into the facility. For example, code 1118 may not only grant the user access to a parking structure, but may also be scanned at access control points through a building (e.g., at doorways) to provide the user access to the building and/or to various areas within the building. Likewise, code 1118 may be scanned at a kiosk (e.g., access terminals 516) or at a front desk of the facility to check-in the user. In this manner, the user's arrival, departure, and movements throughout the building may be tracked via the scanning or transmission of code 1118.

Turning now to FIG. 11D a parking structure navigation interface 1120 is shown, according to some embodiments. Interface 1120 may be displayed in response to the user entering a parking structure, for example. Interface 1120 may present a map or a diagram of the parking structure and may indicate directions for the user to follow to reach an assigned or available parking spot. In some embodiments, a remote parking system that manages available spaces for the parking structure may indicate all available spots, and the best available spot (e.g., closest to the target location or building 10) may be identified. Interface 1120 may direct the user to the available spot.

In some embodiments, the interface 1120 communicates with hardware within the parking structure to direct the user to an assigned or selected parking spot. For example, ceiling lights may be used to illuminate a path to the parking spot, digital display boards may direct the user to the parking spot, or other hardware features of the parking structure may provide guidance. The system can integrate the location system of the user device so that the user's current location is known by the central computing system 602 and directional navigation and coordination with the hardware of the parking structure can be coordinated to guide the user to the parking spot.

In some embodiments, the closest available parking spot to the user's appointment is assigned and the user is directed to the assigned parking spot. In some embodiments, the user selects a valet parking option and the central computing system 602 assigns a valet to meet the user at an assigned entrance that is closest to the appointment. The valet can then meet the user at the closest entrance and valet park the car. This reduces the amount of walking required by user's who desire valet parking. The assignment of an entrance and a valet/parking spot can be integrated with the scheduling system so that the user does not need to manually enter the closest entrance, but rather the central computing system 602 automatically determines the most efficient shortest walking route for the user and assigns the appropriate entrance/parking spot/valet.

In some embodiments, the central computing system 602 coordinates with an autonomous driving system of a user's vehicle and integrates the navigation and parking selection functionality directly into the automated vehicle controls such that the vehicle will recognize an assigned or selected parking spot/entrance/valet and navigate autonomously or semi autonomously as directed by the central computing system 602.

### Automatic Check-In

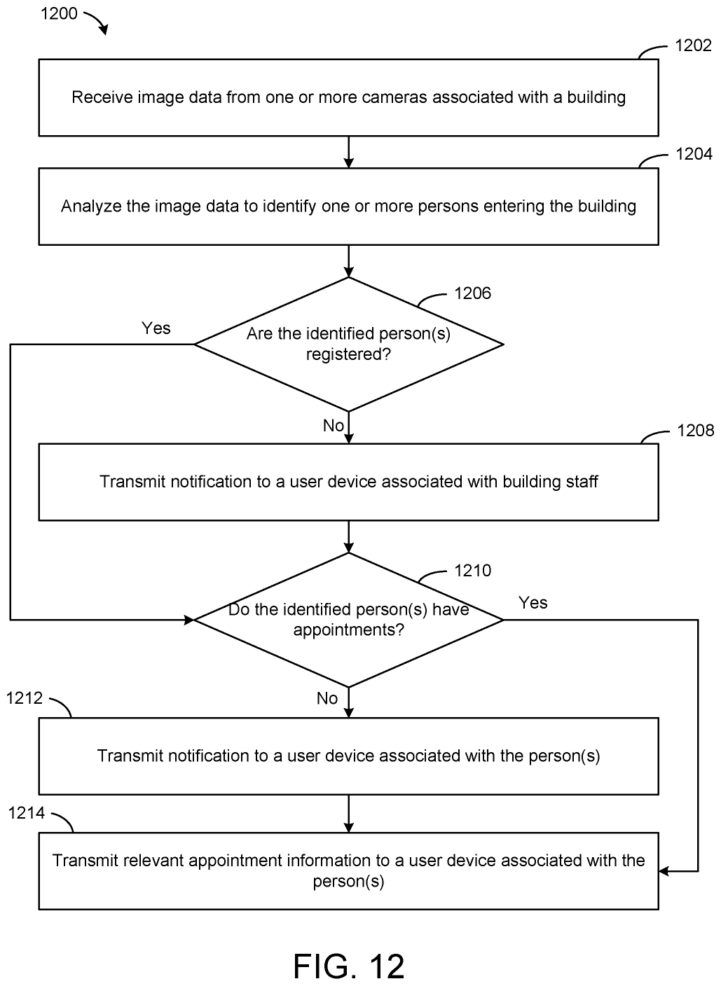

Referring now to FIG. 12, a flow diagram of a process 1200 for automatic check-in based on facial recognition is shown, according to some embodiments. Process 1200 may be implemented by system 600 and more particularly may be implemented by central computing system 602. Advantageously, process 1200 can automate at least portions of check-in procedures that may traditionally have required a large amount of user intervention (e.g., from a building employee). It will be appreciated that certain steps of process 1200 may be optional and, in some embodiments, process 1200 may be implemented using less than all of the steps.

Advantageously, automatically identifying and checking-in building occupants can reduce wait times (e.g., for customers or patients), increase efficiency, and can even increase building security. For example, many hospitals include waiting rooms where patients wait for appointments or procedures. Over time, appointment schedules may back up, causing patients to wait an increasing amount of time for their appointments, which can in turn lead to an increased risk of pathogen transfer (e.g., due to sick patients sneezing or coughing) and patient discomfort, and can lead to overcrowding of the hospital and/or waiting rooms. Accordingly, in combination with the other systems and processes described herein, the automatic check-in process can help to ensure that building occupants (e.g., patients) are only in the building when necessary (e.g., for an appointment).

At step 1202, image data is received from one or more cameras associated with a building. In particular, image data may be received from one or more cameras (e.g., security devices 512) positioned at or near an entrance of the building (e.g., building 10). The one or more cameras may be configured to capture video and/or still images, thereby capturing images of persons entering and/or exiting the building.

At step 1204, the image data is analyzed to identify one or more persons entering the building. The image data may be analyzed by any suitable neural network or artificial intelligence (AI), such as any type of convolutional neural network (CNN). The image data may be input to the neural network or AI, and an output of the neural network or AI may be the identification of one or more persons within the image data. For example, as a patient enters a hospital, security cameras at the front entrance of the hospital may capture image data of the patient, which is then processed by a neural network or AI to identify the patient.

At step 1206, a user records database (e.g., user records 724) or other similar database is queried to determine whether the identified persons are registered with system 600. In particular, identifying the persons may additionally include accessing a database to compare captured and/or processed images of the persons with a reference image of the persons, such as a profile picture or an image taken of the persons during initial registration with the building (e.g., hospital) or an application associated with the building (e.g., application 912).

In the event that one or more of the identified person are not registered (e.g., the identified persons do not have an account established), or in the event that one or more persons cannot be identified, process 1200 may continue to step 1208, where a notification is transmitted to a user device associated with building staff. The notification may include a processed image of the unidentified or unregistered person, and may indicate other details such as when the person entered the building, what entrance the person used, etc. This notification may prompt the building staff to locate the person so that the person can be registered for access to the building. In a hospital, for example, staff may be able to quickly identify persons that are not registered patients such that the unregistered persons can be quickly registered, thereby decreasing wait times. Additionally, notifying building staff of unregistered persons can improve security.

At step 1210, in response to identifying registered persons and/or in response to a new customer or patient being registered, a schedule database (e.g., part of user records 724) or other similar database is queried to determine whether the identified and registered persons have upcoming appointments. In some embodiments, appointments within a predetermined time period (e.g., two hours, one day, one week) may be identified. For example, it may be determined whether the person has an appointment scheduled within the next three hours. In this manner, users may be automatically checked-in for upcoming appointments.

However, it may be determined that user with an appointment outside of the predetermined time period may require services or support outside of their scheduled appointment. For example, a patient at a hospital may come in for an appointment early if there are additional complications or symptoms, or if symptoms have gotten worse since the appointment was scheduled. Accordingly, these patients can be quickly identified, which may lead to faster care. In some embodiments, step 1210 may be skipped if the person is identified at steps 1204 and/or 1206 as a visitor or other guest not requiring an appointment, rather than a patient, customer, etc. For example, visitors at a hospital may not require an appointment but may still be identified and automatically checked-in.

If the identified and registered persons do not have appointments, the process 1200 may continue to step 1212, where a notification is transmitted to a user device associated with a person requiring an appointment. In some embodiments, the notification may be transmitted to a mobile device (e.g., a smartphone) associated with the person requiring the appointment, prompting the person to check-in and/or select an upcoming appointment time. In some embodiments, the notification also prompts the person to input details about their visit, such as a reason for the visit. Alternatively, in some embodiments, a notification may be transmitted to building staff identifying the persons that do not have a scheduled appointment. Thus, building staff may be able to quickly identify unscheduled patients, customers, etc., in order to assist these patients, customers, etc., more efficiently.

At step 1214, relevant appointment information is transmitted to a user device associated with the identified and registered persons. In other words, once a person (e.g., a customer, patient, visitor, etc.) is identified, registered, and/or determined to have an upcoming appointment, information such as appointment time, persons associated with the appointment (e.g., assigned doctors or staff), a room number or location, etc., may be transmitted to the person's device (e.g., smartphone, tablet, etc.). This information may also include navigation data (e.g., generated by navigation engine 716) to a particular space within the building, such as a room. Accordingly, the appointment information may be received and/or displayed by the user device, as discussed in detail below with respect to FIGS. 13A and 13B. In some embodiments, a notification may also be transmitted to a user device of the identified person (e.g., via text message, email, audio recording, etc.), alerting the person when the check-in process is complete.

Referring now to FIGS. 13A and 13B, example interfaces for an automatic check-in process are shown, according to some embodiments. Turning first to FIG. 13A, a check-in interface 1302 is shown. Interface 1302 may be presented via a user interface of user device 604 in response to the user being identified and/or registered via process 1200 described above. Accordingly, following step 1214 of process 1200, the receipt of appointment information may cause user device 604 to generate and display interface 1302.

Interface 1302 may provide a variety of user information, which can be retrieved from a user database (e.g., user records 724). User information can include a name, address, phone number, email address, etc. Additionally, interface 1302 can include a profile picture 1304, which is an image of the user (e.g., “Mr. Smith”). In some embodiments, interface 1302 can include an “Update Profile” icon 1306 that, when selected, may cause user device 604 to display a secondary interface (e.g., via a pop-up window) that allows the user to change various profile information, such as a name, address, phone number, email address, etc.

In some embodiments, interface 1302 may also include appointment details 1308. Appointment details 1308 may include any information relating to the user's upcoming appointments (e.g., the appointments the user was checked-in for by process 1200). For example, appointment details 1308 may include a calendar element that displays the upcoming appointments via a calendar or in a schedule. Appointment details 1308 may also include information such as an assigned room (e.g., smart room 606). In a hospital, appointment details 1308 may include a name and other details regarding an assigned doctor, nurse, or other staff. Accordingly, any details regarding the user's appointment(s) may be displayed via appointment details 1308. Interface 1302 may include any additional fields or graphical elements, such as a comments field 1310 for entering comments that can be shared with building staff. For example, a hospital patient may enter symptoms they're experiencing ahead of the appointment, thereby reducing appointment time by providing this information to a doctor ahead of time.

Turning now to FIG. 13B, a building navigation interface 1312 may also be presented via user device 604. In response to the user checking-in for an appointment (e.g., automatically via process 1200), interface 1312 may be presented to provide directions through the building (e.g., building 10) to a particular area or room, such as an assigned room for the appointment (e.g., a patient room, a meeting room, etc.). In some embodiments, such as in a hospital, interface 1312 may also be presented to visitor, so that they may quickly and easily navigate to a room assigned to a patient they are visiting.

Interface 1312 may include a 2-dimensional (2D) or 3-dimensional (3D) model of the building that indicate areas and devices within the building, such as identifying rooms, elevators, stairways, exits, etc. Interface 1312 may also indicate a route that the user may take through the building to reach a destination (e.g., a particular room). In some embodiments, interface 1312 may include step-by-step directions for aiding navigation through the building. The user may also be able to edit the route/destination or end navigation via interface 1312. In some embodiments, user device 604 may continuously determine a current location in the building based on WiFi, Bluetooth®, RFID, or other similar types of signals. Thus, interface 1312 may continuously update as the user navigates through the building.

### Smart Room Functionality

Referring now to FIGS. 14A-14C, example interfaces for controlling various smart room functions are shown, according to some embodiments. In particular, FIG. 14A includes an example comfort interface 1402 for controlling various comfort parameters of a smart room (e.g., smart room 606), and FIG. 14B includes an example control interface 1414 for controlling various other room and system parameters. These interfaces may be presented to a user occupying or assigned to a particular room, such as smart room 606. For example, a hospital patient may be provided authorization to access interfaces 1402 and 1414 for the particular room they are assigned to, and for the duration of their stay.

Turning first to FIG. 14A, interface 1402 is shown to include a room identification element 1404. Element 1404 may display room information, such as a room number and a room location within the building. In some embodiments, interface 1402 and/or element 1404 may also include an “Assistance” icon or an “Emergency” icon that a user can select to transmit an alert to one or more other users, such as building employees. In a hospital, for example, a patient may select the “Assistance” icon to call a nurse to their room.

Interface 1402 also includes a plurality of control elements 1406, which the user can adjust various room parameters. In some embodiments, selecting a particular element 1406 may cause interface 1402 to be dynamically modified, to display additional options related to the selected element 1406. As shown, for example, the user has selected “Lights” to adjust parameters relating to lighting within the room. However, the user may select “Temperature” to adjust temperature setpoints in the room, or to adjust other climate control parameters. “Blinds” may be selected to open or close automated/motorized blinds (e.g., blinds 806) to control the amount of ambient light entering the room. It will be appreciated that any other elements 1406 may be included for controlling other aspects of a smart room.

Having selected “Lights,” the user may be presented with a variety of light control options for the smart room. In particular, interface 1402 may include an icon that allows the user to turn on or off all of the lights in the room. Interface 1402 also includes a “Main Light” submenu that allows the user to adjust an intensity and/or a color of the main room lights. Likewise, a “Reading Light” submenu allows the user to adjust an intensity of a designated reading light (e.g., a bedside lamp). The user can also turn on or off a night lamp or other light source. In some embodiments, each of these lighting parameters, along with parameters for lighting, blinds, etc., may be limited by one or more constraints. For example, the user may only be able to adjust a room temperature to a minimum or maximum threshold temperature, ensuring that the user does not set the room temperature too high or too low, thereby affecting adjacent spaces. In this example, the user may be limited to a temperature range of 67-74° F.

Turning now to FIG. 14B, interface 1414 may present various other options for a user. In some embodiments, a user may select a “Schedule” icon to view a calendar or other display that presents upcoming appointments or events. For example, a patient at a hospital may select “Schedule” to view upcoming surgeries or doctor visits. A “Staff” icon may present the user with information relating to assigned building staff (e.g., doctors, nurses) and/or may present a staff directory so that the user may contact various building staff. The user may select a “Visitors” icon to view and/or edit visitor information. For example, display area 1418 may present visitors that are approved to visit the user, or upcoming scheduled visits.

In some embodiments, as shown in FIG. 14C, display area 1418 is dynamically modified to display relevant information related to the selected icon 1416. As shown, for example, selecting “Visitors” icon may cause display area 1418 to present a list 1420 of approved visitors. In this case, the user may be able to view the names and profile pictures of approved visitors, and may also edit and/or remove visitors. Additionally, the user made add visitors to the preapproval list by select an icon 1422.

Interface 1414 also includes a “Request Food” icon that can be selected to request a meal, or to change a meal time. For example, a hospital patient may request food outside of a normal meal time by selecting this icon. “Television Controls” may cause display area 1418 to present a virtual remote, or other controls for controlling a television or display (e.g., display 808). “Music” may allow the user to play music or sounds over speakers positioned throughout the room. It will be appreciated that the number and type of icons 1416 or controls that can be presented via interface 1414 is not intended to be limited by those described herein, but that other icons or functions may also be included.

Referring now to FIG. 15, an example interface 1502 for requesting visitor access to a smart room (e.g., the smart room 606) is shown, according to some embodiments. In some embodiments, interface 1502 may be presented when a visitor or non-employee of a building, such that the visitor can input information to request visitor access. In a hospital, for example, a visitor for a patient (e.g., a family member) may be able to configure a visitor profile via interface 1502.

Interface 1502 is shown to include visitor information, including a profile picture 1504, a name, address, phone number, email address, etc. The visitor may be able to update their visitor profile, such as to change their name, address, etc., by selecting an “Update Visitor Profile” icon 1506. The visitor may also select a “Request Access” icon 1508 for requesting access to a particular area of the building, such as a patient's room. In some embodiments, a visitation request may also require the visitor to populate one or more request fields 1510 with information relating to the requested visit. For example, the visitor may indicate the name of a patient they are visiting, along with a requested visit date and time. Accordingly, the visit may be preemptively scheduled to reduce wait times when arriving at the building.

Interface 1502 also includes an approval list 1512, which may display information such as a list of building occupants or spaces that the visitor is approved to access. An additional data field 1514 may also display other visit information. For example, data field 1514 may display a visitation schedule for the visitor, a schedule for the patient, patient information such as room number, etc. Accordingly, the visitor may be able to determine, based on data field 1514, whether the patient is available for a visit at specific times. In some embodiments, visitor requests entered via interface 1502 may be transmitted to a user device associated with the building occupant (e.g., patient) for additional approval.

Referring now to FIG. 16, an example interface 1602 for controlling visitor access to a smart room (e.g., the smart room 606) is shown, according to some embodiments. In particular, interface 1602 may be presented via information panel 814 or another display device positioned outside of a room of the building. Interface 1602 may present a variety of room information, such as a room type and identifier (e.g., a room number), current room occupants, etc. In a hospital, for example, interface 1602 may present a profile picture of the patient occupying the room, as well as information such as the patient's name, an attending doctor or nurse, etc.

In some embodiments, a user may select a “Visitor Check-In” icon 1604 to check-in for a prescheduled or unscheduled visit. In response to a selection of icon 1604, or any of the other icons described below, a display area 1610 may update to display relevant information. For example, when a visitor checks-in, display area 1610 may present an interface similar to interface 1502 described above, allowing the visitor to input information. When a visitor checks in, a notification may also be sent to a user device associated with the room occupant(s).

Selecting “View Schedule” icon 1606 may cause display area 1610 to present a schedule for the room and/or for an occupant of the room. Using a meeting room as an example, display area 1610 may present a schedule that indicates blocks of time that the room is reserved. Likewise, for a patient room in a hospital, display area 1610 may present a schedule for the patient indicating meal times, surgeries, scheduled therapy, etc. Selecting “Staff Access” icon 1608 may present a variety of staff-only functions that may be accessed by inputting authentication information. For example, a nurse may enter log-in credentials and/or may swipe a security badge to access staff-only functions, which allow the staff member to view additional information via interface 1602, or allow the staff member to change parameters.

### Configuration of Exemplary Embodiments

The construction and arrangement of the systems and methods as shown in the exemplary embodiments are illustrative only. Although only a few embodiments have been described in detail in this disclosure, many modifications are possible (e.g., variations in sizes, dimensions, structures, shapes and proportions of the elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

The present disclosure contemplates methods, systems and program products on any machine-readable media for accomplishing operations. The embodiments of the present disclosure can be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures show a specific order of method steps, the order of the steps may differ from what is depicted. Also two or more steps can be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the connection steps, processing steps, comparison steps and decision steps.

1. A building management system (BMS) of a building for controlling a healthcare facility, the BMS comprising:
one or more processing circuits comprising one or more memory devices configured to store instructions thereon that, when executed by one or more processors, cause the one or more processors to:
receive inputs from building devices;
determine a state-of-mind (SOM) score of a patient based on the inputs using a learning model; and
control one or more of the building devices based on the SOM score while maintaining a temperature, a humidity, and a pressure within a compliance standard within the building.

2. The BMS of claim 1, wherein the one or more memory devices are further configured to store instructions thereon that, when executed by the one or more processors, cause the one or more processors to:
determine the SOM score based on visual information, audio information, nurse call information, thermostat information, and occupancy information.

3. The BMS of claim 1, wherein the one or more memory devices are further configured to store instructions thereon that, when executed by the one or more processors, cause the one or more processors to:
determine the SOM score based on electronic medical records information.

4. The BMS of claim 1, wherein the one or more processing circuits include one or more processing circuits located on-premises and comprising one or more on-premises memory devices, and one or more processing circuits located off-premises and comprising one or more off-premises memory devices; and
wherein the one or more off-premises memory devices are configured to store instructions thereon that, when executed by the one or more processors, cause the one or more processors to:
receive confidential information; and
train the learning model using the received confidential information; and
wherein the one or more on-premises memory devices are configured to store instructions thereon that, when executed by the one or more processors, cause the one or more processors to:
receive the trained learning model;
receive the inputs from in-room devices; and
implement the learning model.

5. The BMS of claim 4, wherein the one or more on-premises memory devices are configured to store instructions thereon that, when executed by the one or more processors, cause the one or more processors to:
control the one or more room devices automatically based on the SOM score.

6. The BMS of claim 4, wherein the one or more on-premises memory devices are configured to store instructions thereon that, when executed by the one or more processors, cause the one or more processors to:
correlate the received inputs using the learning model;
update the SOM score using the correlated inputs;
determine control actions for the one or more room devices automatically that are predicted to increase the SOM score.

7. The BMS of claim 6, wherein the one or more on-premises memory devices are configured to store instructions thereon that, when executed by the one or more processors, cause the one or more processors to:
determine modified control actions based on the determined control actions and the compliance standard.

8. The BMS of claim 7, wherein the modified control actions include coordination of a heating, ventilation, and air conditioning (HVAC) system, a lighting system, and a blinds system to improve the SOM score and maintain the compliance standard.

9. The BMS of claim 1, wherein the one or more memory devices are further configured to store instructions thereon that, when executed by the one or more processors, cause the one or more processors to:
determine a number of visitors over a specified period of time based on the inputs from the building devices; and
control one or more room devices automatically including a nurse call system to prompt a visit based on the number of visitors over a specified period of time.

10. The BMS of claim 1, wherein the one or more memory devices are further configured to store instructions thereon that, when executed by the one or more processors, cause the one or more processors to:
determine a number of visitors over a specified period of time based on the inputs from the building devices; and
push a notification to an identified user device to request a point of contact based on the number of visitors over a specified period of time.

11. The BMS of claim 1, wherein the one or more memory devices are further configured to store instructions thereon that, when executed by the one or more processors, cause the one or more processors to:
determine that the SOM score is less than a threshold; and
adjust the temperature or lighting of the healthcare facility automatically based on the SOM score being less than the threshold to meet a patient preference.

12. The BMS of claim 1, wherein the one or more memory devices are further configured to store instructions thereon that, when executed by the one or more processors, cause the one or more processors to:
receive a data set of historical environmental changes and correlated patient reactions;
build patient profiles based on the data set;
associate the patient with a patient profile;
input the patient profile to the learning model to affect the determination of the SOM score.

13. The BMS of claim 1, wherein the one or more memory devices are further configured to store instructions thereon that, when executed by the one or more processors, cause the one or more processors to:
receive patient feedback; and
update the learning model based on the patient feedback.

14. The BMS of claim 1, wherein the one or more memory devices are further configured to store instructions thereon that, when executed by the one or more processors, cause the one or more processors to:
monitor the SOM score over time;
input a change of the SOM score over time into the learning model; and
control the one or more room devices automatically based of the change of the SOM score over time.

15. The BMS of claim 1, wherein the one or more memory devices are further configured to store instructions thereon that, when executed by the one or more processors, cause the one or more processors to:
receive inputs from room devices including audio data from at least one audio sensor;
analyze the audio data using voice recognition to convert speech from a first occupant of the building into a command; and
controlling the one or more room devices based on the command.

16. A system for providing smart functionality within a room of a healthcare facility, the system comprising:
a sensor array configured to detect occupancy within the room, measure physiological parameters of occupants of the room, and measure climate parameters of the room; and
one or more processing circuits comprising one or more memory devices coupled to one or more processors, the one or more memory devices configured to store instructions thereon that, when executed by the one or more processors, cause the one or more processors to:
continuously monitor a comfort level of a patient associated with the room by:
determining, based on first data received from the sensor array, the comfort level of the patient;
comparing the determined comfort level of the patient with a threshold; and
controlling one or more climate control devices associated with the room based on a determination that the comfort level of the patient exceeds the threshold;
determine a healthcare schedule and a visitor schedule for the patient; and
adjust at least one of a scheduled meal time or lighting within the room based on the healthcare schedule and the visitor schedule.

17. The system of claim 16, further comprising at least one audio sensor configured to detect speech from the occupants of the room, the processors further configured to:
receive first audio data from the at least one audio sensor;
analyze the first audio data using voice recognition to convert speech from a first occupant of the room into a command; and
controlling one or more devices in the room based on the command.

18. A system for controlling one or more devices in a room, the system comprising:
one or more processing circuits comprising one or more memory devices, the one or more memory devices configured to store instructions thereon that, when executed by one or more processors, cause the one or more processors to:
determine a room location of a user device;
display, via a user device, a graphical user interface comprising the room location and a first set of graphical elements associated with the one or more devices in the room;
receive, via the graphical user interface displayed on the user device, a first user input selecting a first graphical element of the first set of graphical elements;
dynamically update the graphical user interface based on the first user input to display a second set of graphical elements associated with the first graphical element, the second set of graphical elements presenting one or more parameters of the one or more devices in the room;
receive, via the graphical user interface displayed on the user device, a second user input indicating a change to at least one of the one or more parameters; and
transmit a control signal to at least one device of the one or more devices based on the second user input.

19. The system of claim 18, wherein the one or more memory devices are further configured to store instructions thereon that, when executed by the one or more processors, cause the one or more processors to:
display the first set of graphical elements including a temperature action, a lighting action, or a blinds action.

20. The system of claim 18, wherein the one or more memory devices are further configured to store instructions thereon that, when executed by the one or more processors, cause the one or more processors to:
display the first set of graphical elements including a schedule action, a visitor action, a staff action, a food action, or an audio/visual action.

21. (canceled)

22. (canceled)
